(19) United States
(12) Patent Application Publication
(10) Pub. No.: US 2010/0168085 A1
(43) Pub. Date: Jul. 1, 2010

(54) Dopamine and agonists and antagonists thereof for modulation of suppressive activity of CD4+CD25+ regulatory T cells

(75) Inventors: Michal Eisenbach-Schwartz, Rehovot (IL); Jonathan Kipnis, Modiin (IL)

Correspondence Address: BROWDY AND NEIMARK, P.L.L.C.;624 NINTH STREET, NW, SUITE 300, WASHINGTON, DC, 20001-5303, US

(73) Assignee: YESA RESEARCH AND DEVELOPMENT CO. LTD. at the Weizmann Institute of Science, Rehovot (IL)

(21) Appl. No.: 10/557,901

(22) PCT Filed: May 23, 2004
(86) PCT No.: PCT/IL04/00441

§ 371 Date: Feb. 28, 2007

Related U.S. Application Data
(60) Provisional application No. 60/472,415, filed on May 22, 2003.

(51) Int. Cl. A61K 31/5513; A61K 31/135; A61K 31/195; A61P 35/04; A61P 37/00; A61P 37/06

(52) U.S. Cl. 514/220; 514/649; 514/567; 514/565

(57) ABSTRACT

Compositions and methods for modulation of the suppressive activity of CD4+CD25+regulatory T cells (Treg) on CD4+CD25− effector T cells (Teff) are provided. An agent selected from: (i) dopamine; (ii) a dopamine precursor; (iii) a D1-R agonist; (iv) a D2-R antagonist; (v) a combination of (i) and (ii); or (vi) a combination of (i), (ii) or (iii) with (iv), down-regulates the suppressive activity of Treg and is useful for treatment of cancer. An agent selected from (i) a dopamine D2-R agonist, (ii) a dopamine D1-R antagonist, and (iii) a combination of (i) and (ii), up-regulates the suppressive activity of Treg and is useful for treatment of an autoimmune disease or for controlling graft rejection in tissue/organ transplantation.

### FIELD OF THE INVENTION

The present invention relates to methods and compositions for modulation of the suppressive activity of CD4⁺CD25⁺ regulatory T cells (Treg) on CD4⁺CD25⁻ effector T cells (Teff), in particular for down-regulation of the Treg suppressive activity by dopamine and agonists and antagonists thereof and their use in the treatment of cancer, and for up-regulation of the Treg suppressive activity by other dopamine agonists and antagonists and their use in the treatment of autoimmune diseases and graft rejection.

Abbreviations: APC: antigen-presenting cells; BSA: bovine serum albumin; CNS: central nervous system; CSPG: chondroitin sulfate proteoglycans; CTLA-4: cytotoxic T lymphocyte-associated antigen receptor 4; DA: dopamine; D-R: a dopamine receptor; D1-R: dopamine receptor type 1; D2-R: dopamine receptor type 2; ERK: extracellular signal-regulated kinase; FITC: Fluorescein isothiocyanate; IL: interleukin; MDC: macrophage-derived chemokine; PBS: phosphate-buffered saline; PE: phycoerythrin; SDF-1: stromal-derived factor-1; Teff: effector T-cells; TGF-β: transforming growth factor-β; Treg: regulatory T-cells.

### BACKGROUND OF THE INVENTION

It is becoming increasingly clear that the body, to protect itself against tumor growth or CNS neurodegeneration, needs to elicit an autoimmune response against self-antigens associated with tumors (Dummer et al., 2002) or against self-antigens residing in the site of neurodegeneration (WO 99/60021; Moalem et al., 1999; Mizrahi et al., 2002; Schori et al., 2001a, 2001b; Kipnis et al., 2002b), respectively.

Normally, autoimmunity is suppressed by naturally occurring regulatory CD4⁺CD25⁺ T cells (Treg) (Shevach et al., 2001; Sakaguchi et al., 1995). Therefore, to elicit the desired autoimmune response for anti-tumor therapy or for protection of CNS neurons at risk of degeneration, the Treg-imposed suppression must be alleviated. Depletion of Treg promotes survival of neurons after CNS insults (Kipnis et al., 2002a) and boosts spontaneous anti-tumor autoimmunity (Sakaguchi et al., 2001).

Treg-imposed suppression is a multi-factorial process, involving cell-to-cell contacts (Nakamura et al., 2001) and the activity of soluble factors, which presumably include IL-10 (Sundstedt et al., 2003) and TGF-β (Piccirillo et al., 2002). Studies have shown that the suppressive activity of Treg can be inhibited by addition of exogenous IL-2 (Thornton and Shevach, 1998), or blocking of CTLA-4 (Nakamura et al., 2001; Takahashi et al., 2000), or activation of the newly discovered glucocorticoid-induced TNF-α receptor (GITR) (McHugh et al., 2002; Shimizu et al., 2002).

Some key adhesion molecules are more abundant on the surfaces of Treg than of effector (CD4⁺CD25⁻) T cells (Teff) (Kohm et al., 2002). The ability of Treg to enter tissues might help prevent autoimmune disease progression. In fighting off neurodegeneration or cancer, however, the presence of Treg is a liability. Compounds capable of reducing the trafficking ability (adhesion and migration) of Treg, or their suppressive activity, or both, might therefore be promising candidates for therapy against both cancer and CNS insults. As a corollary, compounds capable of up-regulating the inhibitory or trafficking activity of Treg, or both, might be potential candidates for therapy against autoimmune diseases. A fine balance would then be needed in order to fight off the conditions leading to tumor development or neuronal degeneration without creating conditions that foster autoimmune diseases. Up to now, however, no physiological compounds have been discovered that can control the activity of Treg.

In an attempt to identify physiological compounds potentially capable of controlling the Treg activity as needed, we postulated that since stress- or pain-related physiological compounds are increased after CNS injury (Thiffault et al., 2000; Malcangio et al., 2000; Rothblat and Schneider, 1998), one or more of them might transmit an early signal to Treg, with consequent reduction of the latter's trafficking or suppressive activity or both. We reasoned that likely candidate compounds might be key neurotransmitters such as dopamine, norepinephrine, serotonin, and substance P, all of which have been shown to participate in interactions between the brain and the immune system (Swanson et al., 2001; Edgar et al., 2002).

### Dopamine and Dopamine Agonists and Antagonists

Dopamine (3,4-dihydroxyphenylethylamine or 3-hydroxytiramine) is a catecholamine formed in the body by the decarboxylation of dopa (3,4-dihydroxyphenylalanine) and acts as a neurotransmitter in the CNS. Inside the brain, dopamine acts as a neurotransmitter within the synapse of the nerve cell, and outside the brain (or more specifically outside the blood-brain barrier), it acts as a hormone (like most neurotransmitters) and affects the constriction/dilation of blood vessels. Low-dose dopamine (0.5-3.0 μk/kg/min) infusion is used in hospitals in the treatment of acute renal disease/failure (reviewed in Saxena, 2002). The hydrochloride salt of dopamine (Inotropin) is used intravenously for treatment of hypotension, septic shock and severe congestive heart failure such as in cardiogenick shock.

In Parkinson's disease, a progressive degenerative disease caused principally by the degeneration of the dopaminergic cells in the substantia nigra pars compacta, there is consequent loss of dopamine terminals in the striatum. Since dopamine taken orally is rapidly degraded in the intestine and blood and it does not penetrate from the blood into the brain, the most widely used treatment for Parkinson's disease is pharmacotherapy, mainly by dopamine replacement, administering the precursor L-dopa (levodopa) that is converted to dopamine in the blood and in the brain. The effectiveness of L-dopa is maximized by combination with a medicine such as carbidopa, which blocks the conversion of L-dopa to dopamine in the blood only, thus transporting more L-dopa into the brain, where it is converted to dopamine.

Due to the side effects of the treatment with L-dopa or with the combination L-dopa/carbidopa, dopamine agonists have been developed or are in development for the treatment of Parkinson's disease and other diseases or conditions in which dopamine is involved. Contrary to levodopa, that is converted to dopamine in the body, the dopamine agonists mimic the activity of dopamine by directly activating the dopamine receptor rather than by replacing dopamine as levodopa does.

The receptors for dopamine are primarily found in the striatum. There are at least five subtypes of dopamine receptors, called D1 through D5; the D1 and D5 subtypes belong to the dopamine receptor type 1 family and are referred to as “D1-like” or “D1-R” while the D2, D3, and D4 belong to the dopamine receptor type 2 family and are referred to as “D2-like” or “D2-R”. The receptors are grouped in this manner because of the common properties of the receptor effects.

The different dopamine agonists may have affinity to both D1 and D2 families, albeit with different strength, or they may be specific to the D1 or the D2 family or to one of the receptors within one of the families.

Dopamine agonists having varying activities at the different dopamine receptors are known, or being investigated, that exhibit subtly different effects. Some of the dopamine agonists in use for treatment of Parkinson's disease include apomorphine (D1 and D2 agonist), the ergoline derivatives bromocriptine (D2 agonist), lisuride (D2 agonist), pergolide (D2/D3 strong agonist), and cabergoline (D2 agonist), and the non-ergoline derivatives ropinirole (D2 agonist) and pramipexole (D2/D3 agonist). Bromocriptine and quinpirole protected cortical neurons from glutamate toxicity via the phosphatidylinositol 3 kinase cascade (Kihara et al., 2002). Other dopamine agonists under investigation include the D1 agonists dihydrexidine (DHX, the first high affinity full D1 dopamine receptor agonist), SKF-38393, SKF-81297, and SKF-82958, and the D2 agonists quinpirole, LY 172555, PPHT and quinelorane. SKF-38393 and quinpirole had neuroprotective effects against malonate-induced lesion in the rat striatum, a model of focal ischemial (Fancellu et al., 2003), and in a Parkinson model (Olsson et al., 1995).

Besides their use in the treatment of Parkinson's disease, some dopamine agonists have been proposed for different indications. The D2-R agonists bromocriptine, lisuride, cabergoline, and pergolide have been shown to suppress prolactin secretion and can be used as prolactin inhibitor and in the treatment of pituitary tumors secreting prolactin (usually benign tumors) including macroprolactinomas (Liuzzi et al., 1985; Kleinberg et al., 1983; Colao et al., 1997). Bromocriptine and cabergoline lower serum growth hormone levels in acromegaly patients and can be used for treatment of acromegaly. U.S. Pat. No. 5,744,476 discloses the D2-R agonist dihydrexidine either alone or together with levodopa or with a D2-R agonist, for raising extracellular brain acetylcholine levels to improve cognition in a human having senile or presenile dementia associated with neurodegeneration.

Dopamine has been disclosed to selectively and strongly inhibit the vascular and permeabilizing activities of vascular permeability factor/vascular endothelial growth factor (VPF/VEGF) and to be a candidate for antiangiogenesis therapy (Basu et al., 2001). The D2-R agonists mentioned above for use in prolactinomas also inhibit VEGF and were proposed for antiangiogenic therapy (Goth et al., 2003).

Dopamine antagonists have been developed for several indications, particularly D2 antagonists such as sulpride, spiperone, haloperidol, spiroperidol, clozapine, olanzapine and sertindole for use as antipsychotic agents. Clozapine has also been disclosed for controlling dyskinesias in people with severe Parkinson's disease (Durif et al., 1997).

WO 03/037247 of the same applicant of the present application discloses a method of regulating activity of a T-cell population, the method comprising exposing the T-cell population with a molecule selected capable of regulating a Dopamine receptor activity or the expression of a gene encoding a Dopamine receptor of T-cells of the T-cell population, thereby regulating Dopamine mediated activity in the T-cell population. The method is indicated for treating or preventing a T-cell related disease or condition characterized by abnormal T-cell activity by administration of Dopamine and specific Dopaminergic receptor functional analogs and, more particularly, upregulating Dopamine analogs such as 7-OH-DPAT, (D3/D2 receptor agonist), SKF 38393 (D1-R agonist), quinpirole (D2-R agonist), and PD-168077 (D4-R agonist).

Reference is made to copending International Patent Application No. PCT/IL2004/ . . . entitled “Dopamine and agonists and antagonists thereof for treatment of neurodegenerative diseases” filed by applicant at the Israel PCT Receiving Office (RO/IL) on the same date, the contents thereof being explicitly excluded from the scope of the present invention.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### SUMMARY OF THE INVENTION

The present invention relates, in one aspect, to a method for modulating the suppressive effect of CD4⁺CD25⁺ regulatory T cells (Treg) on CD4⁺CD25⁻ effector T cells (Teff), which comprises administering to an individual in need an agent selected from the group consisting of dopamine, a dopamine precursor, a dopamine agonist, a dopamine antagonist, and a combination thereof.

In one embodiment, the invention relates to a method for down-regulating the suppressive effect of Treg on Teff, said method comprising administering to an individual in need an agent that down-regulates the suppressive activity of Treg on Teff, wherein said agent is selected from the group consisting of:

(i) dopamine or a pharmaceutically acceptable salt thereof;

(ii) a dopamine precursor or a pharmaceutically acceptable salt thereof;

(iii) an agonist of the dopamine receptor type 1 family (D1-R agonist) or a pharmaceutically acceptable salt thereof;

(iv) an antagonist of the dopamine receptor type 2 family (D2-R antagonist) or a pharmaceutically acceptable salt thereof;

(v) a combination of (i) and (ii); and

(vi) a combination of (i), (ii) or (iii) with (iv);

provided that said individual in need is not being treated for a neurodegenerative condition, disorder or disease.

According to this embodiment, the invention provides a method for treatment of cancer, including primary solid and non-solid tumors and metastases thereof.

In another embodiment, the invention relates to a method for up-regulating the suppressive effect of Treg on Teff, said method comprising administering to an individual in need an, agent that up-regulates the suppressive activity of Treg on Teff, wherein said agent is selected from the group consisting of: (i) a dopamine D2-R agonist, (ii) a dopamine D1-R antagonist, and (iii) a combination of (i) and (ii). According to this embodiment, the invention provides a method for treatment of an autoimmune disease or for controlling graft rejection in tissue/organ transplantation.

In another aspect, the present invention relates to a pharmaceutical composition for treatment of cancer comprising a pharmaceutically acceptable carrier and an agent that down-regulates the suppressive activity of Treg on Teff, wherein said agent is selected from the group consisting of: (i) dopamine; (ii) a dopamine precursor; (iii) a D1-R agonist; (iv) a D2-R antagonist; (v) a combination of (i) and (ii); or (vi) a combination of (i), (ii) or (iii) with (iv).

In a further aspect, the present invention relates to a pharmaceutical composition for treatment of an autoimmune disease or for controlling graft rejection in tissue/organ transplantation. comprising a pharmaceutically acceptable carrier and an agent that up-regulates the suppressive activity of Treg on Teff, wherein said agent is selected from the group consisting of: (i) a dopamine D2-R agonist, (ii) a dopamine D1-R antagonist, and (iii) a combination of (i) and (ii).

In still another aspect, the present invention relates to the use of an agent that down-regulates the suppressive activity of Treg on Teff for the manufacture of a pharmaceutical composition for treatment of cancer, wherein said agent is selected from the group consisting of: (i) dopamine; (ii) a dopamine precursor; (iii) a D1-R agonist; (iv) a D2-R antagonist; (v) a combination of (i) and (ii); and (vi) a combination of (i), (ii) or (iii) with (iv),

In still a further aspect, the present invention relates to the use of an agent that up-regulates the suppressive activity of Treg on Teff for the manufacture of a pharmaceutical composition for treatment of an autoimmune disease or for controlling graft rejection in tissue/organ transplantation, wherein said agent is selected from the group consisting of: (i) a dopamine D2-R agonist, (ii) a dopamine D1-R antagonist, and (iii) a combination of (i) and (ii).

In yet another aspect, the present invention relates to an article of manufacture comprising a container containing an agent that down-regulates the suppressive activity of Treg on Teff and instructions for the use of said agent for treatment of cancer, wherein said agent is selected from the group consisting of: (i) dopamine; (ii)' a dopamine precursor; (iii) a D1-R agonist; (iv) a D2-R antagonist; (v) a combination of (i) and (ii); or (vi) a combination of (i), (ii) or (iii) with (iv).

In yet a further aspect, the present invention relates to an article of manufacture comprising a container containing an agent that up-regulates the suppressive activity of Treg on Teff and instructions for the use of said agent for treatment of an autoimmune disease or for controlling graft rejection in tissue/organ transplantation, wherein said agent is selected from the group consisting of: (i) a dopamine D2-R agonist, (ii) a dopamine D1-R antagonist, and (iii) a combination of (i) and (ii).

### BRIEF DESCRIPTION OF THE FIGURES

FIGS. 1a-1d show that dopamine (DA) reduces the suppressive activity mediated by CD4⁺CD25⁺ regulatory T cells (Treg). Proliferation of effector T cells (Teff, a CD4⁺CD25⁻ population) was assayed by incorporation of [³H]-thymidine into Teff co-cultured with naturally occurring Treg. Recorded values are from one representative experiment out of three and are expressed as means±SD of four replicates. (1a) Treg were activated by incubation for 24 h with anti-CD3 antibodies in the presence of mouse recombinant interleukin (mrIL)-2. Incubation of the activated Treg for 2 h with dopamine (10⁻⁵ or 10⁻⁷ M) prior to their co-culturing with Teff reduced their suppression of Teff compared to that obtained with Treg not exposed to dopamine. (1b) Dopamine (10⁻⁵, 10⁻⁷ or 10⁻⁹ M) added to freshly purified Treg. Dopamine (10⁻⁵ M and 10⁻⁷ M) had a similar effect on activity of naïve Treg to that of activated Treg, whereas the effect of dopamine at 10⁻⁹ M on Treg-mediated suppression was not significant. (1c) Activation of Treg for 96 h, followed by the addition of dopamine (10⁻⁵ M) for 2 h at the end of activation, significantly reduced the suppressive activity of Treg on Teff. Incubation of Teff with dopamine (10⁻⁵ M) for 2 h did not affect their susceptibility to Treg-induced suppression. (1d) Addition of norepinephrine (NE) (10⁻⁵ or 10⁻⁷ M) to Treg for 2 h after their activation for 24 h did not affect the suppressive activity of Treg. Significant differences between groups were analyzed by Student's t-test (p<0.001). In all experiments: Teff—50×10³ cells (const.); Treg—from 3×10³ to 50×10³ cells.

FIGS. 2a-2m show the molecular mechanism underlying the effect of dopamine on Treg. (2a) The inhibitory effect of dopamine on the suppressive activity of Treg was mimicked by SKF-38393, a specific agonist of the D1-type family. The D2-type agonist quinpirole did not alter the effect of dopamine on Treg. SCH 23390, a specific D1-type antagonist, wiped out the dopamine effect on the suppressive activity of Treg. Each experiment was performed at least five times and representative results are shown. (2b) Incubation of Treg or Teff with dopamine did not cause apoptosis, as shown by propidium iodide (PI) staining for DNA content and FACS analysis of Treg and Teff, 48 h after their incubation for 2 h with dopamine. (2c) Staining for apoptosis with annexin V for phosphatidylserine on a surface membrane. No increase in annexin V-labeled cells was detected upon incubation of Treg with dopamine (middle panel) or with the D1-type agonist SKF-38393 (right panel). (2d, 2e) Semi-quantitative RT-PCR analysis for D1-R and D5R and TGF-β1 expression. mRNA was extracted from freshly isolated Teff and Treg, incubated for 2 h with or without dopamine, and subjected to semi-quantitative RT-PCR The results of one representative experiment out of five are shown (2d). The housekeeping gene β-actin was used for quantitative analysis of the PCR products. Results are expressed by mean±SEM of 3 independent experiments (2e). (2f) Quantitative real-time PCR using primers for D1-R and D5-R to verify the differences in the expression of dopamine receptors on Teff and Treg. The results presented in the figure are arbitrary units and are from one representative experiment of three performed. (2g, 2h) Semi-quantitative RT-PCR for D2-R, D3-R and D4-R expression. mRNA was extracted from freshly purified Teff and Treg. The housekeeping gene β-actin was used for quantitative analysis. The results of one representative experiment out of five are shown. (2i) Representative micrographs of D1-R-immunoreactive T cells using fluorescence and confocal microscopy. Also shown are micrographs stained with Hoechst and visualized by fluorescence microscopy. D1-R-immunoreactivity was observed in Treg but not in Teff. (2j) Expression of CTLA-4. Treg were activated for 24 h, then incubated for 2 h with dopamine or SKF-38393 (control cells were activated but were not incubated with either dopamine or SKF-38393; note, different cell preparations were used for each treatment and therefore the controls used for each treatment were not the same), and were stained 24 h later for CTLA-4 on cell surfaces. CTLA-4 expression was reduced after exposure to dopamine or to SKF-38393. Representative results of one of five independent experiments with each treatment are shown. (2k) Production of IL-10. Treg were activated for 24 h with anti-CD3 and IL-2 in the presence of lethally irradiated splenocytes (APCs) and then for an additional 2 h with dopamine. Conditioned media were collected after 24, 48, or 72 h of culture and were assayed for IL-10 using a sandwich ELISA. At any given time, significantly less IL-10 was detected in media conditioned by dopamine-treated Treg than in media conditioned by Treg not exposed to dopamine. Statistical significance was verified using a student's T-Test analysis (**, p<0.01; *, p<0.05). The results shown are of one of three independent experiments, performed at each time point. (2l) Lack of IL-2 production by Treg. Treg and Teff were activated separately for 48 h with anti-CD3 and anti-CD28 (without mrIL-2) with or without dopamine. Conditioned media were collected after 48 h and subjected to ELISA. Treg with or without dopamine did not secrete detectable levels of IL-2. Production of 11-2 by Teff was not affected by dopamine. (2m) Foxp3 expression in Treg. Treg were activated for 24 h with anti-CD3 and anti-CD28 in the presence of IL-2, then exposed to dopamine for 2 h, washed, and analyzed 30 min later for Foxp3 expression. No changes in Foxp3 were detected after 30 min of dopamine treatment of naïve Treg.

FIGS. 3a-3e show the correlation between activity of Treg and activation state of ERK1/2. (3a) Treg (12×10³, 25×10³ or 50×10³ cells) were activated by incubation for 30 min with anti-CD3 and anti-CD28 antibodies in the presence of IL-2 and in the presence or absence of tyrosine kinase inhibitor (genistein), and were then co-cultured with Teff (50×10³ cells). The suppression of Teff by Treg was significantly reduced in the presence of genistein. (3b) Similarly, incubation of activated Treg (25×10³ or 50×10³ cells) with the specific MEK inhibitor PD98059, which inhibits the ERK1/2 signaling pathway, almost completely abolished their suppression of Teff (50×10³ cells). (3c, 3d) Western blot analyses of Treg lysates after activation for 20 min with anti-CD3 and anti-CD28, in the presence or absence of dopamine (3c) or SKF-38393 (3d). After activation, the amounts of phospho-ERK1/2 (pERK1, pERK2) seen in Treg are larger than in Teff (3c), but are reduced by dopamine (3c) or by SKF-38393 (3d). Dopamine did not cause a significant change in phospho-ERK1/2 levels in Teff (c); (3e) Quantitative analysis of phospho-bands using NIH Image 1.62.

FIGS. 4a-4i show that dopamine alters the adhesive and migratory activities of Treg. (4a) Treg and Teff were activated for 24 h with anti-CD3 and anti-CD28 and were then incubated, with or without dopamine (10⁻⁵-10⁻⁹M), for 2 h. In the absence of dopamine, adhesion of Treg to the CSPG matrix was significantly stronger than that of Teff. Incubation with dopamine significantly reduced the adhesion of Treg in a concentration-dependent manner. The effect of dopamine on Treg adhesion could be mimicked by SKF-38393, a specific agonist of the D1-type family. The dopamine effect was blocked by SCH-23390, a D1-type antagonist. Dopamine did not significantly alter the adhesion of Teff. A Mann-Whitney nonparametric test was used for statistical analysis. (4b) In the absence of dopamine, adhesion of Treg to fibronectin was only slightly (but still significantly) stronger than that of Teff. However, dopamine did not significantly alter the adhesion of either Treg or Teff. A Mann-Whitney nonparametric test was used for statistical analysis. (4c) Treg were activated for 30 min in the presence or absence of the ERK1/2 signaling pathway inhibitor PD98059, and then subjected to an adhesion assay towards MDC. Adhesion of Treg incubated with PD98059 was significantly weaker than that of control Treg cells. (4d) CD44 expression in Treg and in Teff. FACS analysis showed that significantly larger amounts of CD44 are expressed in Treg than in Teff. After incubation with dopamine, CD44 expression was significantly decreased in Treg, but was not affected in Teff. (4e) The total population of purified CD4⁺ T cells was subjected to a migration assay towards SDF-1 or MDC. The percentage of CD4⁺CD25⁺ T cells in the total population after migration towards CCL22 (MDC) was significantly higher than in the original population. Exposure of Treg to dopamine significantly decreased their migration towards MDC. Migration of Treg towards SDF-1 was not significantly affected by exposure to dopamine. A Mann-Whitney nonparametric test was used for statistical analysis. (4f, 4g) Migration of purified Treg towards MDC was significantly decreased after incubation of Treg with dopamine. Treg in the lower (post-migration) chamber were collected and counted by FACS for a defined time period after staining for membrane CD4 marker. Values are representative results of the FACS analysis (4f) and mean number of cells from triplicates of the same experiment are shown in (4g). (4h, 4i) Semi-quantitative RT-PCR for CCR-4 expression in Treg and Teff. mRNA was isolated from Teff and Treg, incubated for 2 h with or without dopamine. The PCR products were quantified (4i) relative to a housekeeping gene (β-actin). Results of one representative experiment are shown (4h). Each experiment was performed in triplicate and repeated at least three times. ***, p<0.01; **, p<0.01.

FIG. 5 shows that systemic injection of dopamine increases neuronal survival after optic nerve crush injury. Balb/c mice were injected with dopamine (0.4 mg/kg) immediately after being subjected to a partial crush injury of the optic nerve. Two weeks later their retinas were excised and the numbers of surviving neurons determined (see Materials and Methods). Significantly more neurons survived in dopamine-injected mice than in vehicle-injected controls (p<0.01; Student's t-test). Bars represent mean numbers of retinal ganglion cells (RGC)/m² of the retina. Each experiment was performed twice; n=6-8 mice in each group. A two-tailed Student's t-test was used for statistical analysis; ***, p<0.001; **, p<0.01.

FIGS. 6a-6b show that exposure of Treg to dopamine in vitro reduces their suppressive activity in vivo. (6a) Neuronal survival was significantly worse in Balb/c mice that were inoculated (immediately after their exposure to a toxic excess of intraocular glutamate) with activated Treg than in Teff-inoculated mice. Neuronal loss is expressed as a percentage of the number of neurons in untreated glutamate-injected controls. Neuronal survival in Balb/c mice that were exposed to a toxic excess of intraocular glutamate and then treated with activated Treg that were incubated for 2 h with 10⁻⁵ M dopamine before being administered in vivo did not differ from that in vehicle-treated glutamate-injected mice. (6b) Representative micrographs of retinas from mice injected with glutamate and either Teff or Treg. Each experiment was performed twice; n=6-8 mice in each group. A two-tailed Student's t-test was used for statistical analysis; ***, p<0.001.

FIG. 7 shows that the D1-R agonist SKF-38393 improves neuronal survival after CNS insult by glutamate toxicity in mice.

FIG. 8 shows that administration of the D2-R antagonist clozapine alone or together with dopamine increases neuronal survival after glutamate-induced neuronal cell death in mice.

FIGS. 9a-9c show the effect of dopamine and dopamine agonist on tumor growth. (9a) Injection of D1-R agonist SKF-38393 immediately after inoculation of solid M2R tumor cells in mice attenuated significantly tumor development. (9b) Injection of Treg (not exposed to dopamine) immediately after inoculation of solid M2R tumor cells in mice increased incidence and course of tumor development, while injection of Treg exposed to dopamine did not differ from control mice. (9c)) Injection of D1-R agonist SKF-38393 immediately after inoculation of solid M2R tumor cells in SCID mice had no effect on tumor development.

### DETAILED DESCRIPTION OF THE INVENTION

The present invention is based on the surprising finding by the inventors that dopamine blocks the suppressive activity of naturally occurring CD4⁺CD25⁺ cells, which comprise about 10% of the total CD4⁺ population.

The CD4⁺CD25⁺ cells, so-called regulatory T cells (hereinafter designated “Treg”), originally called suppressor T cells, express the transmembrane protein called CD25, that is the α chain of the receptor for IL-2 (Sakaguchi et al., 1995). When activated, Treg begin to secrete large amounts of IL-10 and often some TGF-β as well. Both these lymphokines are powerful immunosuppressants, inhibiting Th1 help for cell-mediated immunity and inflammation and Th2 help for antibody production.

The antigenic peptides recognized by the T-cell receptors of Treg tend to be self-peptides and, perhaps, the major function of Treg cells is to inhibit other T cells (effector cells, hereinafter “Teff”) from mounting an immune attack against self components, namely, to protect the body against autoimmunity. Indeed, it has been confirmed that naturally occurring Treg suppress autoimmunity (Shevach et al., 2001; Sakaguchi et al., 1995).

As mentioned above, recent evidence provided by the present inventors indicate that autoimmunity, that has long been viewed as a destructive process, is the body's endogenous response to CNS injury and its purpose is in fact beneficial (Schwartz and Kipnis, 2001; Yoles et al., 2001). This neuroprotective autoimmunity was shown by the inventors to be inhibited by naturally occurring CD4⁺CD25⁺ cells, that suppressed an endogenous T-cell mediated neuroprotective mechanism to achieve maximal activation of autoimmunity and, therefore, to withstand injury to the CNS (Kipnis et al., 2002a).

It has been recently described that Treg are more prevalent in patients with breast or pancreas cancer than in normal controls. In pancreas tumor-bearing mice the prevalence of Treg increases with tumor progression. Purified Treg were found to suppress proliferation and cytokine secretion of non-Treg (namely, Teff), and depletion of Treg in mice lead to significantly smaller tumors compared to control mice, thus indicating that a combination of Treg depletion followed by vaccination in cancer patients could be feasible (Liyanage et al., 2002).

Several control mechanisms including Treg operate in the organism in order to prevent autoimmunity. These same mechanisms, however, create major obstacles for effective immunotherapy of cancer. Therapeutic efficacy of a tumor cell-based vaccine against experimental B16 melanoma requires the disruption of either of two immunoregulatory mechanisms that control autoreactive T cell responses: the cytotoxic T lymphocyte-associated antigen (CTLA)-4 pathway or the Treg cells. Combination of CTLA-4 blockade and depletion of CD25(+) Treg cells results in maximal tumor rejection. Efficacy of the antitumor therapy correlates with the extent of autoimmune skin depigmentation. The synergism in the effects of CTLA-4 blockade and depletion of CD25(+) Treg cells indicates that CD25(+) Treg cells and CTLA-4 signaling represent two alternative pathways for suppression of autoreactive T cell immunity. Simultaneous intervention with both regulatory mechanisms is therefore a promising concept for the induction of therapeutic antitumor immunity (Sutmuller et al., 2001).

Modulation of Treg cell responses seems to be a critical factor in human immunotherapy. Immunotherapy of melanoma targeting melanocyte differentiation antigens involves the induction of autoimmunity; therefore tumor immunity and autoimmunity are two of a kind.

Treg were shown to be involved in several autoimmune diseases. Thus low numbers of resting CD4(+) CD25(+) T cells in IDDM patients; a subset of T cells shown to have important immunoregulatory functions in abrogating autoimmunities in 3-day thymectomized experimental mice. It seems that multiple immunoregulatory T (Treg) cell defects underlie islet cell autoimmunity leading to IMD in humans and that these lesions may be part of a broad T cell defect (Kukreja et al., 2002). In all their subjects with immune-mediated diabetes—about half, newly diagnosed children and adults, and the rest, adults with long-standing disease—the authors found a deficiency in natural killer T cells (or NK T cells) and CD4+/CD25+ T cells. Together, these are called T regulatory cells (Treg cells), because they regulate the immune system and protect the body from being attacked by its own defenses. These findings have implications for therapy: instead of suppressing the immune system, just the right part of it, the T regulatory cells, should be stimulated. Since these cells are not totally absent from people with immune-mediated diabetes, they might be stimulated to function better.

Although the concept of suppression mediated by T lymphocytes was originally proposed more than 30 years ago, recent studies in animal models of autoimmunity have rekindled interest in the existence of a subset of lymphocytes that specifically suppress immune responses. One population of naturally-occurring or endogenous T suppressor cells can be identified by co-expression of the CD4 and CD25 antigens. These cells suppress the activation of CD4 and CD8 T cells in vitro by an unknown cell-contact dependent mechanism. In vivo, these cells suppress autoimmune disease by both cell contact-dependent and suppressor cytokine-dependent pathways. Although these cells were originally described in the mouse, a population with identical phenotypic and functional properties has been identified in man. Determination of the cellular target and the molecular basis of CD4+CD25+ mediated suppression is a major area of current research. The nature of the physiologic ligand recognized by these cells is also unknown as is the breadth of their T cell repertoire. Suppressor/regulatory T cells have been primarily been shown to inhibit animal models of autoimmune disease. However, recent studies have extended their range of activities to inhibition of tumor immunity, graft rejection, allergic disease, graft versus host disease, and acute and chronic infectious diseases. Enhancement of regulatory T cell function in vivo by pharmacologic means is an useful approach in autoimmunity, allergic disease, and graft rejection. Similarly, inhibition of regulatory T cell function, either transiently or permanently, by pharmacologic manipulation or treatment of animals or man with monoclonal antibodies specific for effector molecules on these cells, might be useful in tumor therapy. By developing protocols for the adoptive immunotherapy of both CD4⁺CD25⁺ T cells that have been expanded in vitro or and for suppressor cells that have been induced in vitro, these cells can be administered to patients with GVHD, graft rejection, and organ-specific and systemic autoimmune disease.

Peripheral tolerance to allogeneic organ grafts can be induced in rodents by treating with non-depleting CD4 and CD8 monoclonal antibodies. This tolerance is maintained by CD4+ T cells with a potent capacity to induce tolerance in further cohorts of T cells (i.e. infectious tolerance). CD4+T-cell subsets against the male transplantation antigen were cloned in vitro. In contrast to Th1 or Th2 clones that elicit rejection, it was found that there is a distinct population of CD4+ T cells that suppress rejection by adoptive transfer (called Treg). In order to identify molecular markers associated with tolerance and gain insights into the mechanisms of action of Treg cells, serial analysis of gene expression was carried out. Genes overexpressed in Treg were identified and compared to Th1 and Th2 cultures and found that some of these correlated in vivo with CD4-induced transplantation tolerance rather than rejection. The genes overexpressed in Treg cultures and within tolerated skin grafts were primarily expressed by mast cells (e.g. tryptophan hydroxylase and FcεR1α), suggesting that regulatory cell activity and this form of tolerance may be associated with a localised but non-destructive form of Th2-like activation and a recruitment of mast cells (Zelenika et al., 2001).

In its broad aspect, the present invention relates to a method for modulating the suppressive effect of Treg on Teff, which comprises administering to an individual in need an agent selected from the group consisting of dopamine, a dopamine precursor, a dopamine agonist, a dopamine antagonist, and a combination thereof.

According to one embodiment, the invention provides a method for down-regulating the suppressive effect of Treg on Teff, which comprises administering to an individual in need an agent selected from the group consisting of: (i) dopamine; (ii) a dopamine precursor: (iii) a D1-R agonist; (iv) a D2-R antagonist; (v) a combination of (i) and (ii); and (vi) a combination of (i), (ii) or (iii) with (iv), wherein said individual is not being treated for a neurodegenerative condition, disorder or disease.

As used herein, the terms “dopamine”, “D1-R agonist” and “D2-R antagonist” are meant to include the compounds themselves as well as their pharmaceutically acceptable salts.

In one most preferred embodiment, the agent is dopamine or a pharmaceutically acceptable salt thereof such as the hydrochloride or hydrobromide salt, and is preferably dopamine hydrochloride.

In another preferred embodiment, the agent is dopamine in combination with its precursor levodopa, optionally in further combination with carbidopa.

In another preferred embodiment, the agent is a dopamine D1-R agonist selected from any such agonist known or to be developed in the future and includes, without being limited to, a D1-R agonist selected from the group consisting of A-77636, SKF-38393, SKF-77434, SKF-81297, SKF-82958, dihydrexidine and fenoldopam. Preferably, the D1-R agonist is SKF-38393 and its hydrochloride salt [(+/−)-1-Phenyl-2,3,4,5-tetrahydro-(1H)-3-benzazepine-7,8-diol.HC1].

In a further preferred embodiment, the agent is a dopamine D2-R antagonist selected from any such antagonist known or to be developed in the future and includes, without being limited to, a D2-R antagonist selected from the group consisting of amisulpride, clozapine, domperidone, eticlopride, haloperidol, iloperidone, mazapertine, olanzapine, raclopride, remoxipride, risperidone, sertindole, spiperone, spiroperidol, sulpride, tropapride, zetidoline, CP-96345, LU111995, SDZ-HDC-912, and YM 09151-2. Preferably, the D2-R antagonist is clozapine.

In a further preferred embodiment, the agent is a combination of dopamine with a dopamine D2-R antagonist, preferably dopamine and clozapine.

In still a further preferred embodiment, the agent is a combination of dopamine D1-R agonist with a dopamine D2-R antagonist, preferably a combination of SKF-38393 and clozapine.

When a combination of compounds is used, the two agents may be administered concomitantly (in mixture) or subsequently to each other.

According to one preferred embodiment, the present invention relates to a method for treatment of cancer, said method comprising administering to a cancer patient an agent that down-regulates the suppressive activity of Treg on Teff, wherein said agent is selected from the group consisting of: (i) dopamine or a pharmaceutically acceptable salt thereof; (ii) a dopamine precursor or a pharmaceutically acceptable salt thereof; (iii) an agonist of the dopamine receptor type 1 family (D1-R agonist) or a pharmaceutically acceptable salt thereof; (iv) an antagonist of the dopamine receptor type 2 family (D2-R antagonist) or a pharmaceutically acceptable salt thereof; (v) a combination of (i) and (ii); and (vi) a combination of (i), (ii) or (iii) with (iv).

According to this embodiment, the method is intended for triggering of tumor regression, stimulation of the natural immunological defense against cancer, and/or inhibition of cancer cell metastasis. It is not intended to include in this definition the angiogenesis therapy of tumors based on the antiangiogenic activity disclosed for dopamine (Basu et al., 2001).

The tumor to be treated according to the invention is a malignant tumor and may be a solid tumor such as, but not limited to, a bladder, brain, breast, cervix, colon, esophagus, head and neck, larynx, liver, lung, melanoma, ovary, pancreas, prostate, renal, stomach, thyroid, uterus, vagina or vocal cord tumor. The tumor may also be a non-solid malignant neoplasm such as a lymphoproliferative disorder selected from multiple myeloma, non-Hodgkin's lymphomas, and a lymphocytic leukemia e.g. chronic lymphocytic leukemia (CLL), prolymphocytic leukemia (PLL), hairy cell leukemia, large granular lymphocyte leukemia, and Waldenstrom's macroglubulinemia.

In another aspect, the invention provides a method for the up-regulation of the suppressive activity of Treg on Teff, said method comprising administering to an individual in need an agent that up-regulates the suppressive activity of Treg on Teff, wherein said agent is selected from the group consisting of: (i) an antagonist of the dopamine receptor type 1 family (D1-R antagonist) or a pharmaceutically acceptable salt thereof; (ii) an agonist of the dopamine receptor type 2 family (D2-R agonist) or a pharmaceutically acceptable salt thereof; and (iii) a combination of (i) and (ii).

As used herein, the terms “D2-R agonist” and “D1-R antagonist” are meant to include the compounds themselves as well as their pharmaceutically acceptable salts.

In one embodiment, the method for up-regulation of the suppressive effect of Treg comprises administration of a dopamine D2-R agonist. The dopamine D2-R agonist may be any such agonist known or to be developed in the future and includes, without being limited to, a D2-R agonist selected from the group consisting of bromocriptine, cabergoline, lisuride, pergolide, pramipexole, quinagolide, quinpirole, quinelorane, ropinirole, roxindole, talipexole, LY 171555 [4aR-trans-4,4-a,5,6,7,8,8a,9-o-dihydro-5n-propy1-2H-pyrazolo-3-4-quinoline. HCl), PPHT [(±)-2-(N-phenylethyl-N-propyl)amino-5-hydroxytetralin] and TNPA [2,10,11-trihydroxy-N-propylnoraporphine]. In another embodiment, the method comprises administration of a dopamine D1-R antagonist. The dopamine D1-R antagonist may be any such antagonist known or to be developed in the future and includes, without being limited to, a D1-R agonist selected from the group consisting of SCH 23390, NNC 756, NNC 0.01-112 and CEE-03-310.

In a further embodiment, the method comprises administration of a combination of a dopamine D2-R agonist and a dopamine D1-R antagonist.

According to one embodiment of the present invention, the method for up-regulation of the suppressive activity of Treg on Teff is directed to an individual suffering from an autoimmune disease.

Thus, the present invention further provides a method for treatment of an autoimmune disease, said method comprising administering to an individual suffering from an autoimmune disease an agent that up-regulates the suppressive activity of Treg on Teff, wherein said agent is selected from the group consisting of (i) a dopamine D2-R agonist or a pharmaceutically acceptable salt thereof, a dopamine D1-R antagonist or a pharmaceutically acceptable salt thereof, and (iii) a combination of (i) and (ii).

The autoimmune disease may be an organ specific or systemic autoimmune disease and includes, without being limited to, Eaton-Lambert syndrome, Goodpasture's syndrome, Grave's disease, Guillain-Barré syndrome, autoimmune hemolytic anemia (AIHA), hepatitis, insulin-dependent diabetes mellitus (IDDM), systemic lupus erythematosus (SLE), multiple sclerosis (MS), myasthenia gravis, plexus disorders e.g. acute brachial neuritis, polyglandular deficiency syndrome, primary biliary cirrhosis, rheumatoid arthritis, scleroderma, thrombocytopenia, thyroiditis e.g. Hashimoto's disease, Sjögren's syndrome, allergic purpura, psoriasis, mixed connective tissue disease, polymyositis, dermatomyositis, vasculitis, polyarteritis nodosa, polymyalgia rheumatica, Wegener's granulomatosis, Reiter's syndrome, Behçet's syndrome, ankylosing spondylitis, pemphigus, bullous pemphigoid, dermatitis herpetiformis, Crohn's disease or uveitis.

In another embodiment, the method for up-regulation of the suppressive activity of Treg on Teff is applied to an individual undergoing tissue transplantation in order to prevent graft rejection.

Thus, the present invention still further relates to a method for controlling graft rejection in an individual undergoing tissue or organ transplantation which comprises administering to said individual an agent that up-regulates the suppressive activity of Treg on Teff, wherein said agent is selected from the group consisting of (i) a dopamine D2-R agonist, a dopamine D1-R antagonist, and (iii) a combination of (i) and (ii).

The transplantation may be of any organ or tissue such as cornea, heart, kidney, liver, lung, pancreas, etc. and the agent will be administered according to protocols used to prevent transplant rejection.

Pharmaceutical compositions for use in accordance with the present invention may be formulated in a conventional manner using one or more physiologically acceptable carriers or excipients. The carrier(s) must be “acceptable” in the sense of being compatible with the other ingredients of the composition and not deleterious to the recipient thereof. The term “carrier” refers to a diluent, adjuvant, excipient, or vehicle with which the drug is administered.

Methods of administration include, but are not limited to, parenteral, e.g., intravenous, intraperitoneal, intramuscular, subcutaneous, mucosal (e.g., oral, intranasal, buccal, vaginal, rectal, intraocular), intrathecal, topical and intradermal routes. Administration can be systemic or local.

As will be evident to those skilled in the art, the therapeutic effect depends at times on the condition or disease to be treated, on the individual's age and health condition, on other physical parameters (e.g., gender, weight, etc.) of the individual, as well as on various other factors, e.g., whether the individual is taking other drugs, etc., and thus suitable doses and protocols of administration will be decided by the physician taking all these factors into consideration.

The invention will now be illustrated by the following non-limiting examples and accompanying figures.

### EXAMPLES

### Materials and Methods

(i) Animals. Inbred adult wild-type and nu/nu Balb/c and C57B1/6 mice were supplied by the Animal Breeding Center of The Weizmann Institute of Science (Rehovot, Israel). All animals were handled according to the regulations formulated by IACUC (Institutional Animal Care and Use Committee).

(ii) Antibodies and reagents. Mouse recombinant IL-2, anti-mouse ζ-CD3 (clone 145-2C11), anti-mouse CTLA-4 (CD152) (clone #63828), and purified rabbit anti-mouse ERK2 antibody were purchased from R&D Systems (Minneapolis, Minn.). Rat anti-mouse phycoerythrin (PE)-conjugated CD25 antibody (PC61) was purchased from Pharmingen (Becton-Dickinson, Franklin Lakes, N.J.). Fluorescein isothiocyanate (FITC)-conjugated anti-CD4 antibody was purchased from Serotec (Oxford, UK). Anti dopamine receptor-1 (D1-R; Cat no 324390) was purchased from Calbiochem (Darmstadt, Germany). The compounds 3-hydroxytyramine (3,4-dihydroxyphenethylamine; dopamine) (H-8502), norepinephrine (A-7257), SKF-38393 (D-047), SCH-23390 (D-054), quinpirole (Q-111), clozapine (C-6305), genistein (G-6649), and PD98059 (P-215) were from Sigma-Aldrich (Rehovot, Israel). The phosphatidylserine detection kit, which includes FITC-labeled annexin V, was purchased from IQ Products (Houston, Tex.). Anti-pERK1/2 FITC-conjugated 1 & 2 phosphospecific antibody was purchased from Biosource International (Camarillo, Calif.). Purified anti-pERK1/2 antibody was the generous gift of Prof. R. Seger from The Weizmann Institute of Science.

(iii) Intravitreal glutamate injection. The right eyes of anesthetized mice were punctured with a 27-gauge needle in the upper part of the sclera, and a 10-μL Hamilton syringe with a 30-gauge needle was inserted as far as the vitreal body. A total volume of 1 μl, of L-glutamate (400 nmol) dissolved in saline was injected into the eye.

(iv) Retrograde labeling of retinal ganglion cells. Mice were anesthetized and placed in a stereotactic device. The skull was exposed and kept dry and clean. The bregma was identified and marked. The designated point of injection was at a depth of 2 mm from the brain surface, 2.92 mm behind the bregma in the anteroposterior axis and 0.5 mm lateral to the midline. A window was drilled in the scalp above the designated coordinates in the right and left hemispheres. The neurotracer dye FluoroGold (5% solution in saline; Fluorochrome, Denver, Colo.) was applied (1 μL, at a rate of 0.5 μL/min in each hemisphere) using a Hamilton syringe, and the skin over the wound was sutured.

(v) Crush injury of the optic nerve in mice. Animals were deeply anesthetized by intraperitoneal injection of Xyl-M® 2% (xylazine, 10 mg/kg; Arendonk, Belgium) and Ketaset (ketamine, 50 mg/kg; Fort Dodge Laboratories, Fort Dodge, Iowa) and subjected to severe crush injury of the intraorbital portion of the optic nerve. The uninjured contralateral nerve was left undisturbed. The optic nerve was crushed 3 days after retrograde labeling of retinal ganglion cells with FluoroGold, as described above (Fisher et al., 2001).

(vi) Preparation of splenocytes. Donor splenocytes from rats (aged up to 10 weeks) were obtained by rupturing the spleen and following conventional procedures. The splenocytes were washed with hypotonic buffer (ACK) to lyse red blood cells.

(vii) FACS analysis of CTLA-4-expressing CD4⁺ T cells. Cells were immunostained according to the manufacturer's instructions, resuspended in 0.4 mL of 1% paraformaldehyde, and analyzed by FACSort (Becton-Dickinson), with 10,000 events scored. In single-color analysis, positive cells were defined as cells with higher immunofluorescence values, on a logarithmic scale, than those of control cells incubated with isotype antibodies as a control. The cells were scored from a region defined according to physical parameters that indicate the size (forward scatter) and granularity (side scatter) of lymphocytes. CD4⁺ lymphocytes were then gated for analysis of CTLA-4 expression.

(viii) FACS analysis of annexin V-positive regulatory T cells. PE-stained CD25⁺ cells were stained for annexin V-FITC according to the manufacturer's instructions. The cells were scored as described above, and CD25⁺ lymphocytes were then gated for analysis of annexin V-positive cells.

(ix) FACS analysis of intracellular labeling of phosphorylated form of ERK. T-cell subpopulations were fixed in 1.5% formaldehyde for 10 min at room temperature, washed, resuspended with vortexing in cold 100% methanol, and incubated for 1 h at 4° C. The cells were then washed with PBS containing 1% bovine serum albumin (BSA) and resuspended in 100 μL of PBS/BSA, and 12.5 μL of anti-phosphoERK1 & 2 (BioSource) was added for 20 min at room temperature. The cells were then washed, resuspended in PBS, and analyzed by FACSCalibur (Becton-Dickinson). As a negative control we incubated the phospho-peptide for 20 min with its antibody and then added the mixture to T cells.

(x) Enzyme-linked immunosorbent assay. Treg or Teff (0.5×10⁶ cells/ml) were cultured for 48 h in the presence of anti-CD3 and anti-CD28. After 48 h the cells were centrifuged and their supernatants were collected and sampled. Concentrations of IL-2 in the samples were determined by the use of sandwich enzyme-linked immunosorbent assay (ELISA) kits (R&D Systems, Minneapolis, Minn.). For detection of secreted IL-10 cells were centrifuged every 24 h and replaced with a fresh medium. Supernatants obtained from cells after 24, 48 and 72 h in culture were subjected to ELISA kit (Diaclone Research, Fleming, France). The plates were developed using a 3,3′,5,5′-tetramethyl-benzidine liquid substrate system (Sigma, St. Louis, Mo.). The reaction was stopped by adding 1M H₃PO₄. Results for each experiment were calculated as the amount of secreted cytokine per 1 ml of sample, after subtraction of the background levels of the medium.

(xi) Depletion of CD25⁺ cells. Splenocytes obtained from wild-type mice were prepared by the standard procedure, and incubated with rat anti-mouse PE-conjugated CD25 antibody and then with anti-PE beads (Becton-Dickinson). The washed splenocytes were subjected to AutoMacs (Miltenyi Biotec, Gladbach, Germany) using the ‘deplete sensitive’ program. Recovered populations were analyzed by FACSort.

(xii) Purification of murine CD4⁺CD25⁺/CD4⁺CD25⁻ T cells. Lymph nodes (axillary, inguinal, superficial cervical, mandibular, and mesenteric) and spleens were harvested and mashed. T cells were purified (enriched by negative selection) on T-cell columns (R&D Systems). The enriched T cells were incubated with anti-CD8 microbeads (Miltenyi Biotec), and negatively selected CD4⁺ T cells were incubated with PE-conjugated anti-CD25 (30 μg/10⁸ cells) in PBS/2% fetal calf serum. They were then washed and incubated with anti-PE microbeads

(Miltenyi Biotec) and subjected to magnetic separation with AutoMACS. The retained cells were eluted from the column as purified CD4⁺CD25⁺ cells. The negative fraction consisted of CD4⁺CD25⁻ T cells. Purified cells were cultured in 24-well plates (1 mL) with T cell-depleted spleen cells as accessory cells (irradiated with 3000 rad) and 0.5 μg/mL anti-CD3, supplemented with 100 units of mouse recombinant IL-2 (mrIL-2; R&D Systems).

(xiii) T cell adhesion. Adhesion of activated CD4⁺CD25⁺ and CD4⁺CD25⁻ T cells to CSPG was analyzed as previously described (23). Briefly, flat-bottomed microtiter (96-well) plates were pre-coated with CSPG (1 μg/well, 40 min, 37° C.). ⁵¹Cr-labeled T cells were left untreated or were pre-incubated (30 min, 37° C.) with dopamine or the specified agonist or antagonist (10⁻⁵M). The cells (10⁵ cells in 100 μL of RPMI containing 0.1% BSA) were then added to the CSPG-coated wells, incubated (30 min, 37° C.), and washed. Adherent cells were lysed and the resulting supernatants were removed and counted in a γ-counter. Results were expressed as the mean percentage of the total population before adhesion of bound T cells from quadruplicate wells for each experimental group.

(xiv) Chemotaxis assay. The migration of T cells across polycarbonate filters (pore size 5 μm, diameter 6.5 mm) towards SDF-1 and MDC (CCL22) was assayed in 24-well Transwell chambers (Costar, Corning, Corning, N.Y.). T lymphocytes (1.67×10⁶ cells/mL) were suspended in RPMI/0.1% BSA, and 150 μL of the cell suspension was added to the upper chamber after incubation with or without dopamine (90 min, 37° C.). Chemokines were added to the lower chamber at concentrations of 1 μg/mL SDF-1 (CytoLab, Israel) and 0.25 μg/mL MDC (R&D Systems). The plates were incubated for 90 min at 37° C. in 9.5% CO₂. T cells that migrated to the lower chambers were collected and stained with anti-CD4 and anti-CD25 antibodies. The numbers of migrating T cells were measured by flow cytometer acquisition for a fixed time (60 s). To calculate specific migration, the number of cells in each subpopulation in the absence of chemokine was subtracted from the number in the corresponding cell subpopulation that migrated in the presence of chemokines. The number of migrating CD4⁺CD25⁺ T cells was calculated as a percentage of the total T cell population before migration. For migration of purified population we used a similar protocol.

(xv) Propidium iodide staining. Cells were fixed in cold ethanol 80% and treated with RNAse. Propidium iodide was then applied, and cell samples were assessed by FACSort.

(xvi) Activation of CD4⁺CD25⁺ regulatory T cells. Purified regulatory T cells (Treg; 0.5×10⁶ cells/mL) were activated in RPMI medium supplemented with L-glutamine (2 mM), 2-mercaptoethanol (5×10⁻⁵ M), sodium pyruvate (1 mM), penicillin (100 IU/mL), streptomycin (100 μg/mL), nonessential amino acids (1 mL/100 mL), and autologous serum 2% (vol/vol) in the presence of mrIL-2 (5 ng/mL) and soluble anti-CD3 antibodies (1 ng/mL). Irradiated (2500 rad) splenocytes (1.5×10⁶ cells/mL) were added to the culture. Cells were activated for 24 or 96 h. In some of the 96-h experiments, fresh dopamine was added to the culture every 24 h during activation.

(xvii) Inhibition assay (co-culturing of Teff with Treg). Naïve effector T cells (Teff; 50×10³ cells/well) were co-cultured with decreasing numbers of activated Treg for 72 h in 96-well flat-bottomed plates in the presence of irradiated splenocytes (10⁶/mL) supplemented with anti-CD3 antibodies. [³H]-thymidine (1 μCi) was added for the last 16 h of culture. After the cells were harvested, their [³H]-thymidine content was analyzed by the use of a γ-counter.

(xviii) Immunocytochemistry. T cells were fixed for 10 min with a mixture (1:1) of methanol and acetone at −20° C., incubated in blocking solution (PBS containing 0.3% Triton-X100 and 1% of normal rabbit serum) for 60 min at room temperature, and then incubated overnight with a specific antibody (dilution 1:1000) in the blocking solution. The T cells were then washed and incubated with the secondary antibody (PE-labeled goat anti-rabbit IgG) for 30 min at room temperature, then washed, and analyzed by fluorescence and confocal microscopy.

(xix) Western blotting. Cells were stimulated for 20 min with anti-CD3 and anti-CD28 antibodies in the presence or absence of dopamine or SKF-38393. Cell lysates were prepared using RIPA lysis buffer (50 mM Tris, pH 8; 0.1% SDS; 0.5% deoxycholate; 1% NP40; 500 mM NaCl; 10 mM MgCl₂) and were then kept on ice for 10 min before being vortexed and centrifuged. Supernatants were collected and 5× sample buffer (containing 25 mM Tris pH 6.8, 2% sodium dodecyl sulfate (SDS), 10% glycerol, 0.1% bromophenol blue, 0.5 M β-mercaptoethanol) was added prior to boiling. Cell extracts were separated by SDS-PAGE (10% polyacrylamide), and blotted onto nitrocellulose. Activated ERK1/2 was detected by probing blots with a 1:30,000 dilution of monoclonal antibody. Total ERK protein was detected by using a 1:10,000 dilution of a polyclonal rabbit antibody. The blots were developed by horseradish peroxidase-conjugated anti-mouse or anti-rabbit Fab and ECL (Amersham). Signals were quantified using NIH Image 1.62.

(xx) Polymerase chain reaction (PCR). Total RNA was purified with the RNeasy Mini Kit (Qiagen, Germantown, Md.) and transcribed into cDNA using poly dT primers. For PCR the following primers were used:

for D1-R:

[SEQ ID NO: 1]

| | |
|---|---|
| sense, | 5′-GTAGCCATTATGATCGTCAC-3′, |

[SEQ ID NO: 2]

| | |
|---|---|
| anti-sense, | 5′-GATCACAGACAGTGTCTTCAG-3′, |
| for D2-R: | |

[SEQ ID NO: 3]

| | |
|---|---|
| sense, | 5′-GCAGCCGAGCTTTCAGGGCC-3′, |

[SEQ ID NO: 4]

| | |
|---|---|
| anti-sense, | 5′-GGGATGTTGCAGTCACAGTG-3′, |
| for D3-R: | |

[SEQ ID NO: 5]

| | |
|---|---|
| sense, | 5′-AGGTTTCTGTCAGATGCC-3′, |

[SEQ ID NO: 6]

| | |
|---|---|
| anti-sense, | 5′-GTTGCTGAGTTTTCGAACC-3′, |
| for D4-R: | |

[SEQ ID NO: 7]

| | |
|---|---|
| sense, | 5′-CACCAACTACTTCATCGTGA-3′, |

[SEQ ID NO: 8]

| | |
|---|---|
| anti-sense, | 5′-AAGGAGCAGACGGACGAGTA-3′, |
| for D5-R: | |

[SEQ ID NO: 9]

| | |
|---|---|
| sense, | 5′-CTACGAGCGCAAGATGACC-3′, |

[SEQ ID NO: 10]

| | |
|---|---|
| anti-sense, | 5′-CTCTGAGCATGCTCAGCTG-3′, |
| for CCR-4: | |

[SEQ ID NO: 11]

| | |
|---|---|
| sense, | 5′-GTGCAGTCCTGAAGGACTTCAAGCTCCACCAG-3′ |

[SEQ ID NO: 12]

| | |
|---|---|
| anti-sense, | 5′-GGCAAGGACCCTGACCTATGGGGTCATCAC-3′, |
| and | |
| for FOXP3: | |

[SEQ ID NO: 13]

| | |
|---|---|
| sense, | 5′-CAG CTG CCT ACA GTG CCC CTA G-3′, |

[SEQ ID NO: 14]

| | |
|---|---|
| anti-sense, | 5′-CAT TTG CCA GCA GTG GGT AG-3′. |

Signals were quantified using a Gel-Pro analyzer 3.1 (Media Cybernetics). Real-time PCR was performed with a LightCycler instrument (Roche Diagnostics, Mannheim, Germany) using FastStart DNA Master SYBR Green 1 kit (Roche, catalog no. 3003230) as described by the manufacturer. The following primers were used for the reactions: For D1-R, the primers listed above. For D5-R: sense, 5′-CCTTTATCCCGGTCCA-3′ [SEQ ID NO: 15], anti-sense, 5′-GATACGGCGGATCTGAA-3′ [SEQ ID NO: 16]; for IL-10 sense, 5′-ACCTGGTAGAAGTGATGCCCCAGGCA-3′[SEQ ID NO: 17], anti-sense, 5′-CTATGCAGTTGATGAAGATGTCAAA-3′ [SEQ ID NO: 18] (Pozzi et al., 2003); for Foxp3; sense, 5′-CAG CTG CCT ACA GTG CCC CTA G-3′ [SEQ ID NO: 19], anti-sense, 5′-CAT TTG CCA GCA GTG GGT AG-3′ [SEQ ID NO: 20].

(xxi) Assessment of mouse retinal ganglion cell survival. Mice were given a lethal dose of pentobarbitone (170 mg/kg). Their eyes were enucleated and the retinas were detached and prepared as flattened whole mounts in 4% paraformaldehyde solution. Labeled cells from 4-6 selected fields of identical size (0.7 mm²) were counted. The selected fields were located at approximately the same distance from the optic disk (0.3 mm) to overcome the variation in RGC density as a function of distance from the optic disk. Fields were counted under the fluorescence microscope (magnification ×800) by observers blinded to the identity of the retinas. The average number of RGCs per field in each retina was calculated.

### Example 1

### Dopamine Reduces the Suppression Imposed by Treg

In this experiment, we examined whether dopamine acts on Treg and alters their suppressive effect on Teff. Suppression of proliferation of Teff, assayed by [³H]thymidine incorporation, was used as a measure of suppressive effect of Treg (Thornton and Shevach, 1998).

Co-culturing of Teff with Treg isolated from naïve mice results in suppression of Teff proliferation. The suppressive potency depends on the Treg/Teff ratio and the state of Treg activation; the suppression is significantly increased, for example, if the Treg are activated before being added to Teff (Thornton and

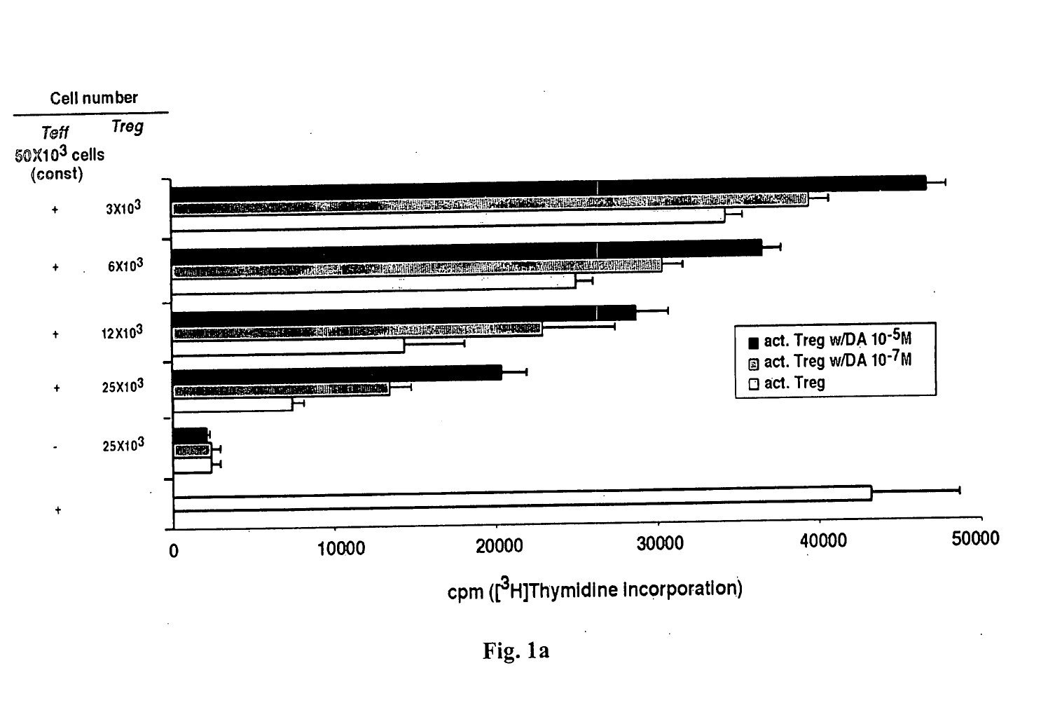

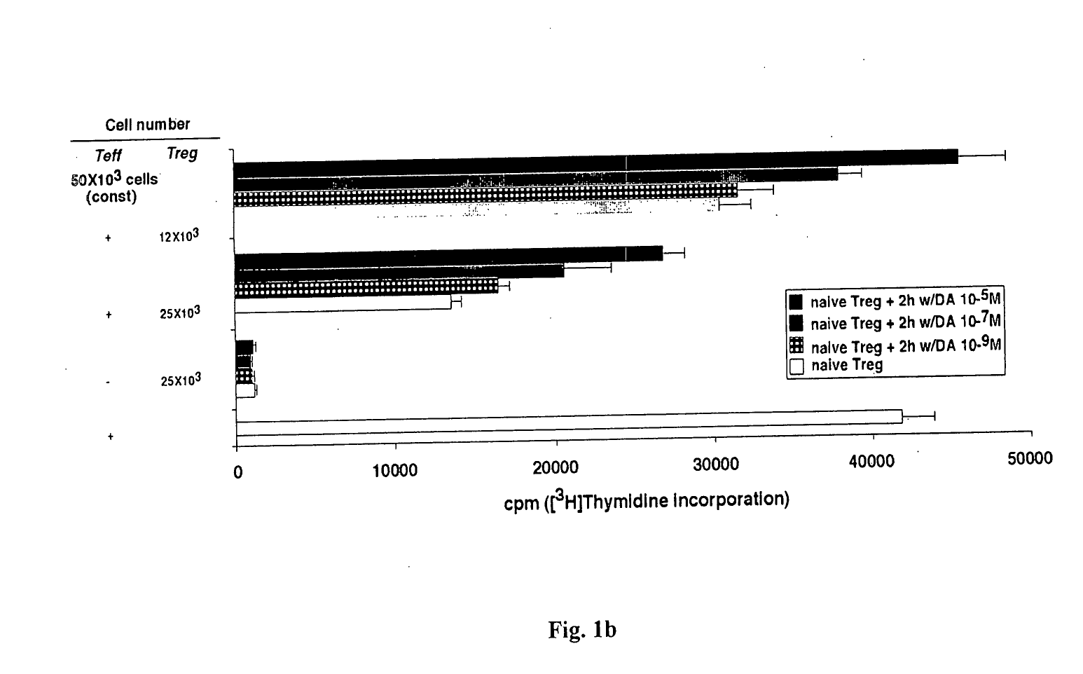

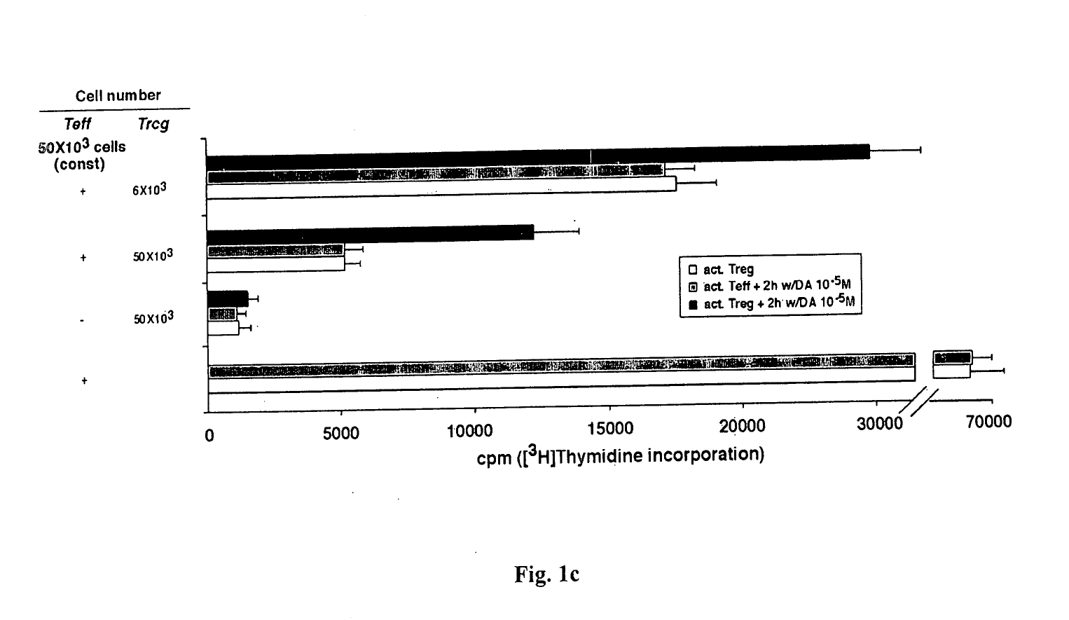

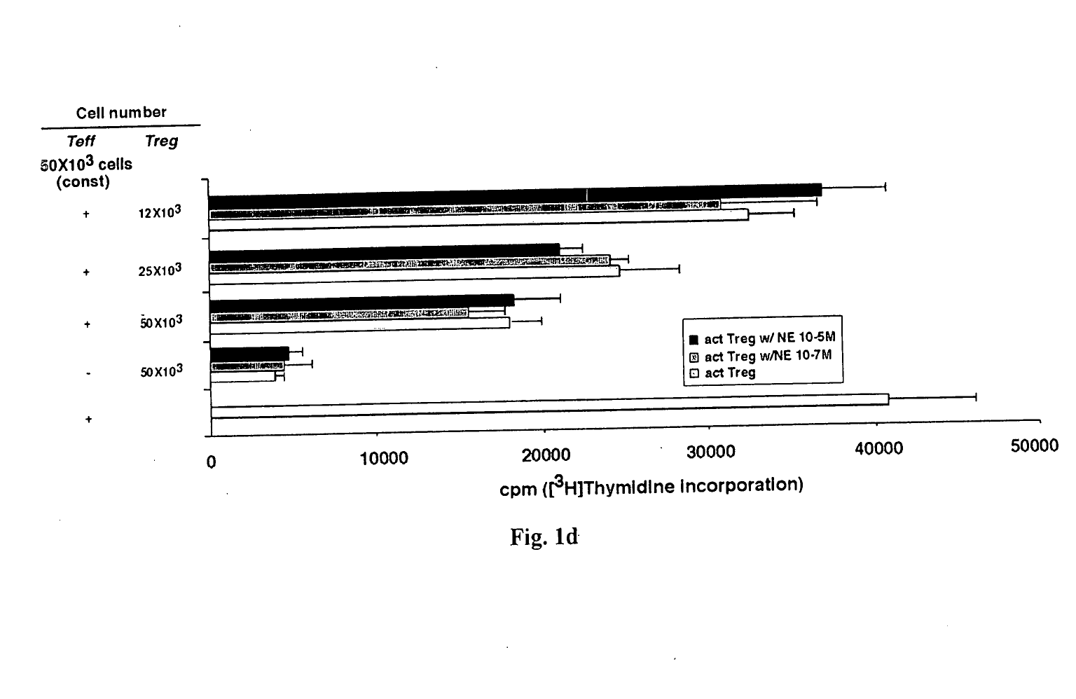

Shevach, 1998, 2000). Inhibition of Treg proliferation, assayed by [³H]thymidine incorporation, can therefore be taken as a measure of the suppressive effect. We examined the ability of major neurotransmitters and neuropeptides (dopamine, norepinephrine, substance P, and serotonin) to alleviate the Treg-induced suppression of Teff in vitro. Each compound was tested at several concentrations. Proliferation of Teff was significantly inhibited by co-cultivation of Teff with naïve Treg or with Treg that had been activated by incubation for 24 h with anti-CD3 antibodies and IL-2 in the presence of antigen-presenting cells (APCs, lethally irradiated splenocytes; FIG. 1). After incubating the activated Treg for 2 h with a neurotransmitter or a neuropeptide, we washed the cells and then co-cultured them with Teff. Proliferation of Teff co-cultured with activated Treg that had been incubated with dopamine (10⁻⁵ M) was more than twofold higher than proliferation in co-culture with activated Treg not incubated with dopamine (FIG. 1a). A significant effect on Treg suppressive activity was also obtained with 10⁻⁷ M dopamine (FIG. 1a), whereas 10⁻⁹ M had no significant effect (data not shown). The inhibitory effect of dopamine at 10⁻⁵ and 10⁻⁷ M on Treg activity was reproduced when freshly isolated (nonactivated) Treg were used (FIG. 1b). At dopamine concentration of 10⁻⁹ M, the obtained effect was slight and not statistically significant (FIG. 1b). It should be noted, however, that the effect of dopamine on Treg suppressive activity was only partial, and that complete blocking was not seen at any of the concentrations tested.

We also examined the effect of dopamine on the activity of Treg that had been activated as described above (FIG. 1a), but for 96 h, and to which dopamine (10⁻⁵ M) was added for 2 h at the end of the activation period, and then washed off before the activated cells were co-cultured with naïve Teff. Again, Teff proliferation was significantly higher in the presence of activated Treg treated with dopamine than in the presence of activated Treg without dopamine (FIG. 1c). A direct effect of dopamine on Teff proliferation was ruled out by incubation of Teff for 2 h with 10⁻⁵ M dopamine, then washing off the dopamine and adding activated Treg without dopamine. The resulting proliferation of Teff did not differ from that seen in cultures of Teff in the absence of dopamine. Moreover, the inhibitory effects of Treg on naïve Teff and on Teff exposed to dopamine were similar (FIG. 1c), indicating that dopamine did not alter the susceptibility of Teff to Treg suppression.

The uptake of thymidine by Teff and the Treg-induced inhibition of such uptake varied from one experiment to another. In all experiments, however, the effect of dopamine on Treg (tested more than 20 times) was consistent, and in most cases the proliferation of Teff co-cultured with Treg treated with dopamine was more than twofold higher than that in the absence of dopamine treatment. The Treg used in this experiment were always obtained from naïve animals, therefore, it is unlikely that they contained any activated effector T cells. The purity of the Treg population used in all experiments was high (between 92% and 98% of the total CD4⁺ population). Moreover, the use of anti-CD25 antibodies to isolate Treg reportedly does not interfere with either the suppressive activity or the state of activation of Treg (Thornton and Shevach, 1998).

In contrast to the effect seen with dopamine, no effect on the ability of Treg to suppress Teff proliferation could be detected when Treg were preincubated with different concentrations of norepinephrine (another member of the catecholamine family; FIG. 1d), substance P (a pain- and stress-related neurotransmitter; data not shown), or serotonin (data not shown).

### Example 2

### The Effect of Dopamine on Treg is Exerted Via Type-1 Family of Dopamine Receptors (D1-R)

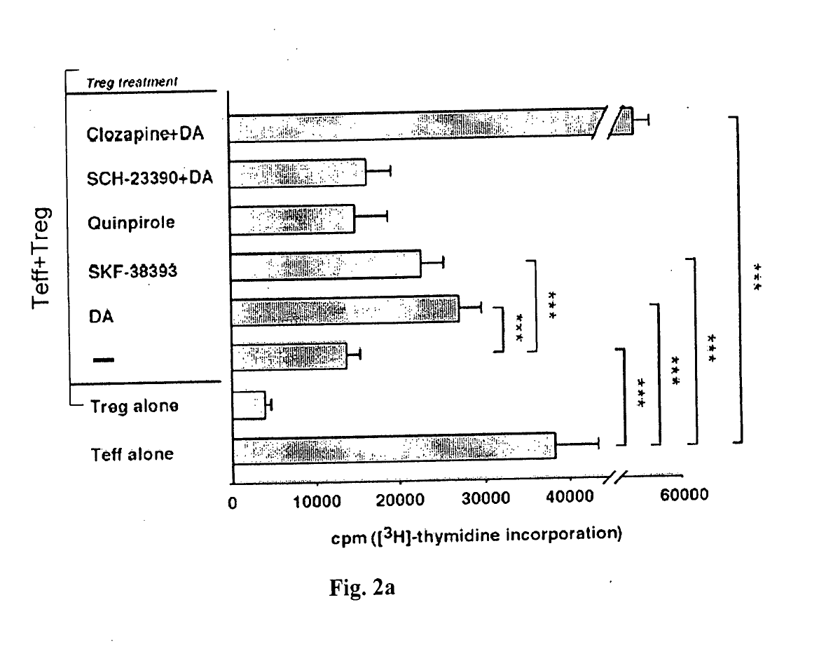

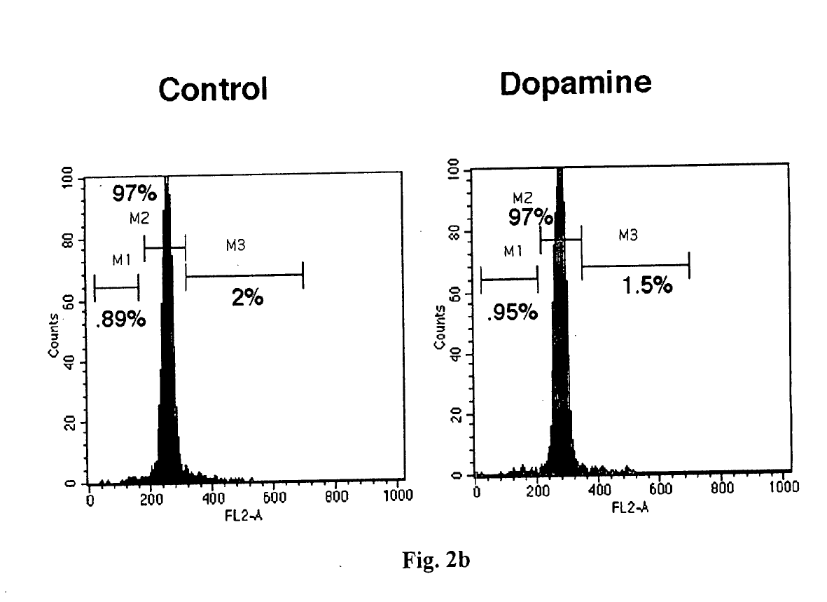

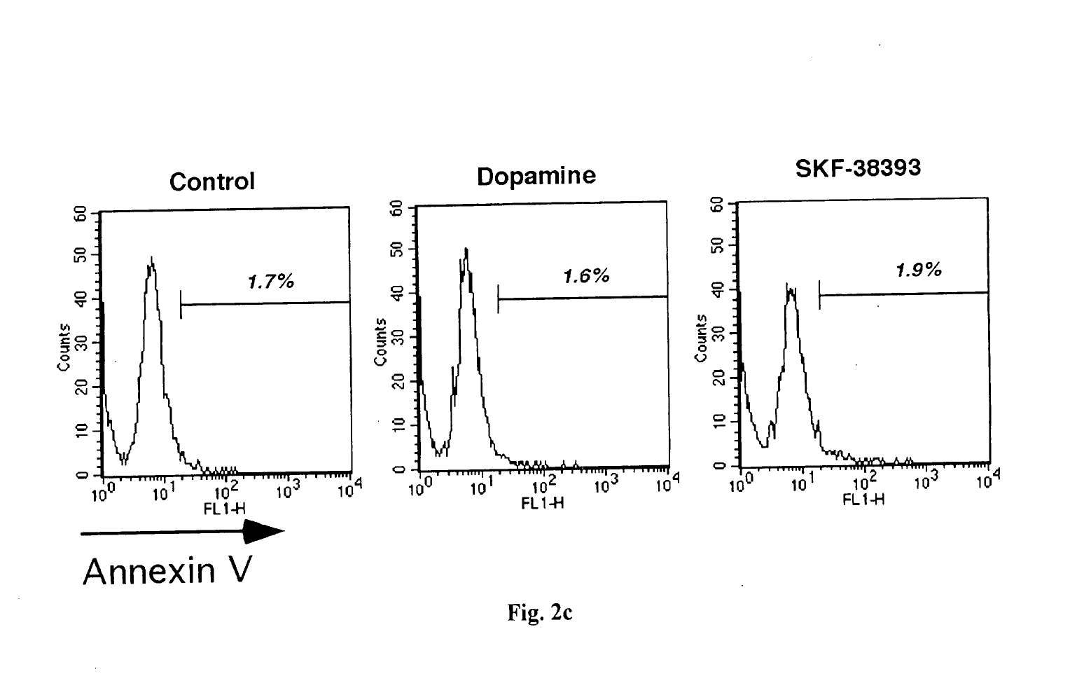

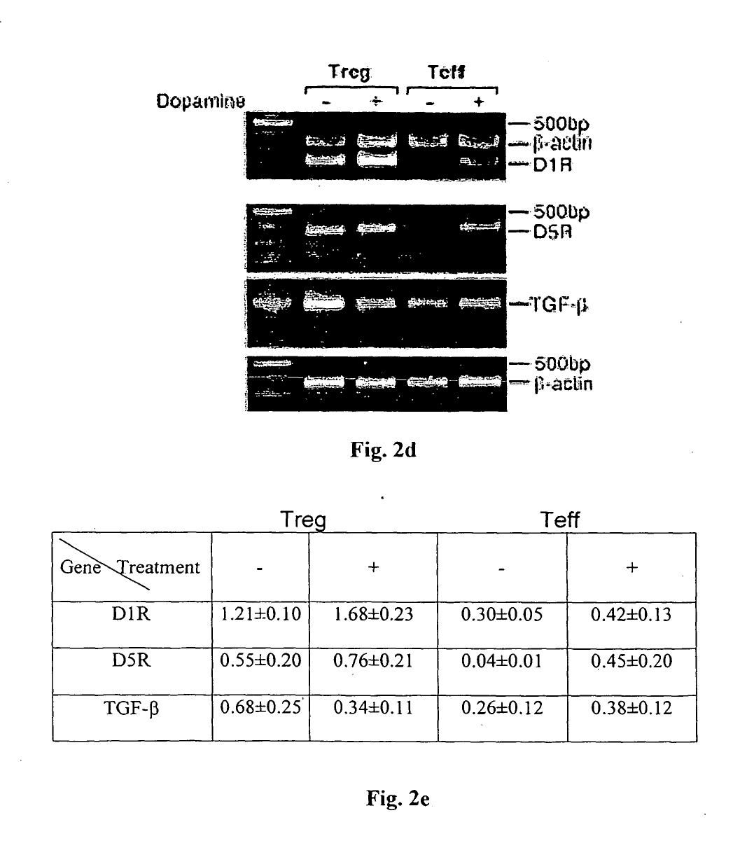

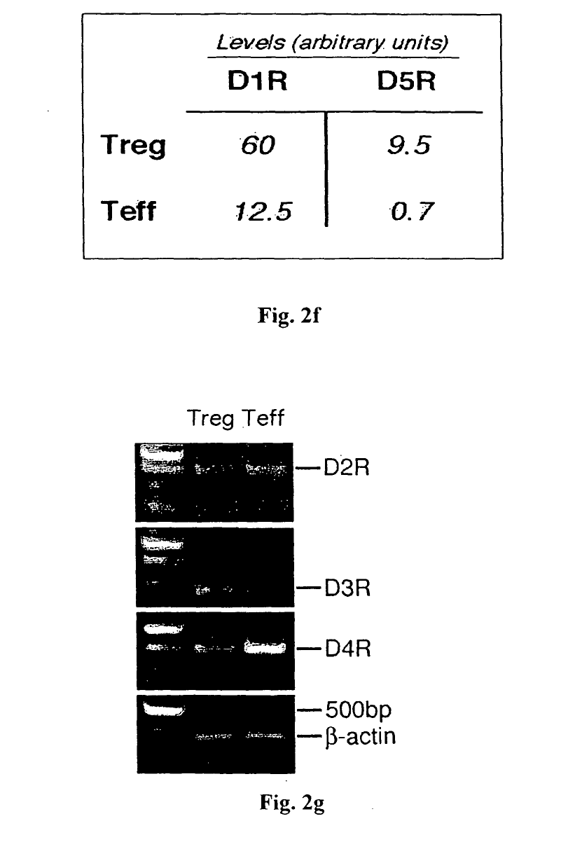

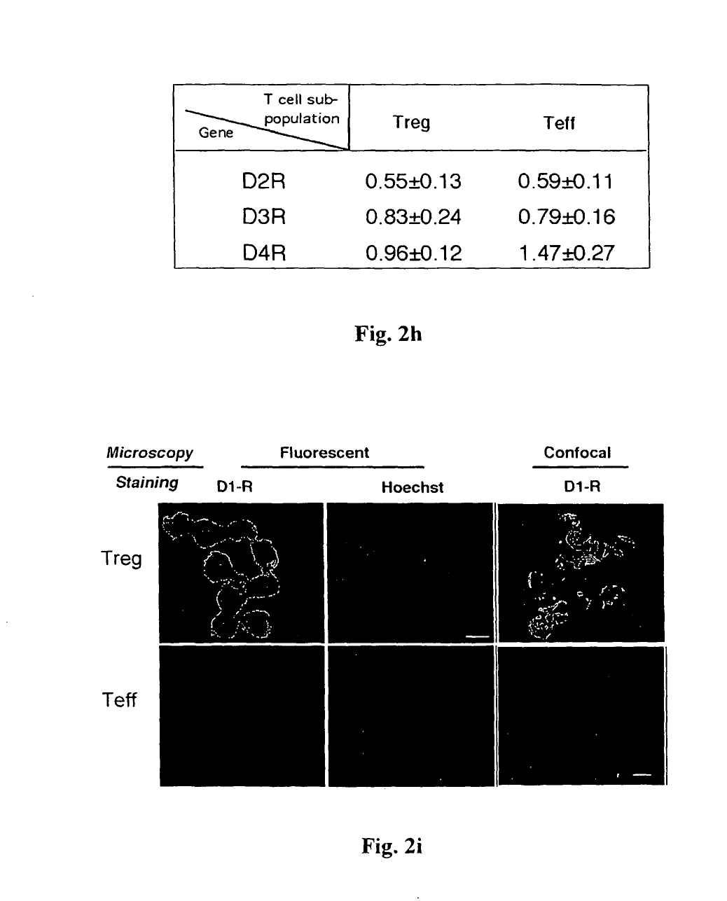

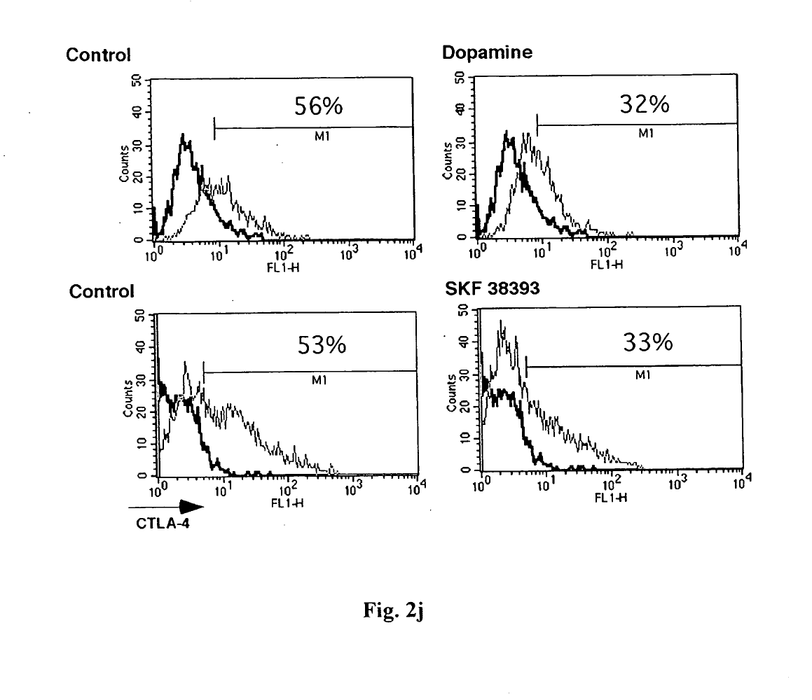

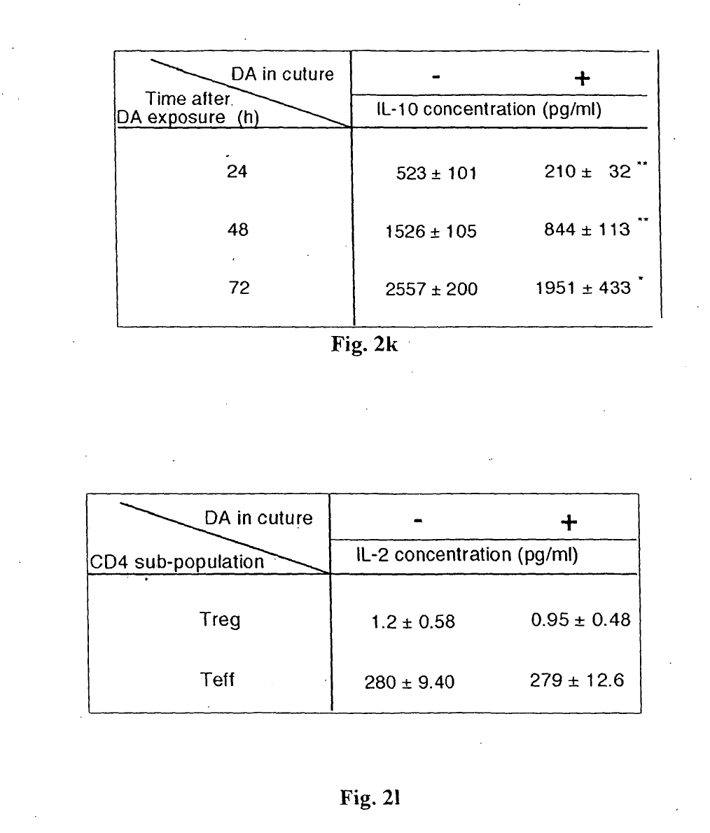

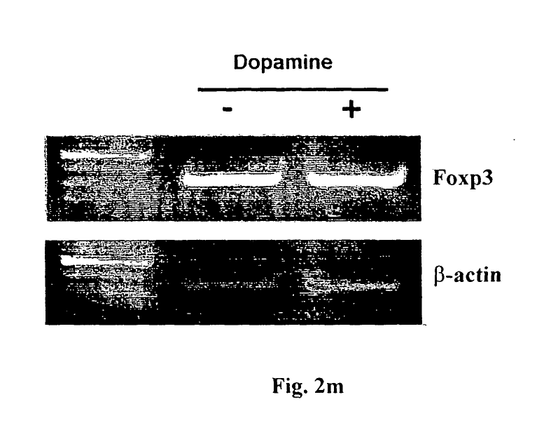

To establish whether the observed effect of dopamine on Treg is exerted through a receptor-mediated pathway, we employed specific agonists and antagonists of dopamine receptors. Incubation of Treg with 10⁻⁵ M SKF-38393, an agonist of the type-1 family of dopamine receptors (consisting of D1-R and D5-R), reproduced the dopamine effect (FIG. 2a). The specific D1-type antagonist SCH-23390 (10⁻⁵M), when added together with dopamine (10⁻⁵M), prevented the dopamine effect, further substantiating the contention that the effect of dopamine on Treg is mediated through the type-1 receptor family. Also in line with this contention was the finding that incubation of Treg with 10⁻⁵ M quinpirole, an agonist of the type-2 family of dopamine receptors (comprising D2-R, D3-R, and D4-R), had no effect on the suppressive activity of Treg. However, clozapine, an antagonist of D2-R, enhanced the dopamine-induced inhibitory effect, resulting in complete blocking of suppression (FIG. 2a).

### Example 3

### Dopamine does not Cause Treg Apoptosis

To exclude the possibility that dopamine exerts its effect by causing the death of Treg, we examined whether dopamine at the concentrations used here cause Treg apoptosis. No signs of apoptosis were detectable in Treg, which, after being incubated with dopamine, were stained with propidium iodide and analyzed for apoptotic cells (sub-G1) by flow cytometry (FIG. 2b). To further verify the absence of apoptotic death in Treg, after incubating Treg with dopamine we stained them for phosphatidylserine with annexin V. Again, we could not detect any signs of apoptosis in Treg beyond the background levels seen in the absence of dopamine

(FIG. 2c). Thus, the reduction in Treg activity after their encounter with dopamine or a related agonist, evidently results not from the death of Treg, but rather from alteration of their behavior.

### Example 4

### Expression of Dopamine Type-1 and Type-2 Receptors in Treg and in Teff

Since dopamine reduced the suppressive activity of Treg on Teff but did not alter the susceptibility of Teff to suppression by Treg, we examined the possibility that Teff and Treg express different subtypes or different amounts of the relevant dopamine receptors. This was done by assaying the expression of the dopamine type-1 receptors, D1-R and D5-R, in Treg and Teff, in the absence and in the presence of dopamine (incubation of the cells for 2 h with 10⁻⁵ M dopamine). PCR assays showed that Treg expressed significantly more D1-R and D5-R transcripts (4-fold and 14-fold, respectively) than Teff (FIGS. 2d, 2 e).

Incubation of the cells with dopamine did not significantly alter the number of D1-R transcripts in either Treg or Teff. The number of D5-R transcripts in Treg were also unchanged, but in Teff they showed a 10-fold increase, reaching numbers similar to those in Treg. In contrast, within the limit of error, such incubation did not change the number of D5-R transcripts in Treg. Dopamine did not significantly alter the number of D1-R transcripts in either Treg or Teff (FIG. 2d, 2e).

Because the suppressive effect of Treg on Teff is partly due to transforming growth factor (TGF)-β1 (Nakamura et al., 2001), we measured whether exposure of Treg to dopamine affects the level of TGF-β1 expression. Transcripts encoding TGF-β1, which might contribute to the suppressive effect of Treg, were decreased in Treg after dopamine treatment (FIGS. 2d, 2e), suggesting that the observed blocking of suppression results, at least partially, from a decrease in expression of TGF-β1.

To further verify the differences in expression of dopamine receptors by Teff and Treg, we carried out real-time PCR, which showed that the amounts of D1-R and D5-R in Treg were 5-fold and 13-fold higher, respectively, than in Teff (FIG. 2f).

We also used PCR to assay the expression of dopamine type-2 family receptors, namely D2-R, D3-R, and D4-R in Treg and Teff. Although the expression of D4-R was somewhat more abundant in Teff than in Treg, the difference between the expression of each of these receptors in the two T-cell subpopulations was not significant (FIGS. 2g, 2h), further substantiating our finding that the preferential effect of dopamine on Treg is through the family of D1-type receptors. To verify that the difference in D1-R between Treg and Teff observed at the transcript level is manifested also at the protein level, we subjected the cells to immunocytochemical analysis. D1-R-immunoreactivity was detected in naïve Treg, but not in naïve Teff (FIG. 2i).

### Example 5

### Dopamine Affects CTLA-4 Expression and IL-10 Production by Treg

To gain further insight into the mechanism whereby dopamine affects Treg activity we examined CTLA-4, a molecule characteristic of Treg (Im et al., 2001). Expression of this molecule was slightly but consistently decreased upon exposure of Treg to dopamine. A similar effect on CTLA-4 expression was obtained with the D1-type specific agonist SKF-38393 (FIG. 2j).

Another molecule that participates in the suppressive activity of Treg is IL-10 (Maloy et al., 2003). It was therefore of interest to measure the production of IL-10 by Treg after their exposure to dopamine. Media collected after incubation of Treg with dopamine (10⁻⁵ M) for 24 h, 48 h and 72 h showed a significant decrease in the amount of IL-10 at all time points examined (FIG. 2k).

Dopamine did not, however, alter the anergic state of Treg; production of IL-2 was not detected in Treg that had been incubated in the presence of dopamine, as verified by ELISA for a secreted cytokine in media conditioned for 48 h by activated Treg (FIG. 21). Teff, as expected, secreted IL-2, the level of which was not affected by dopamine (FIG. 21). It should be noted that activation of both T cell populations was carried out in the absence of mrIL-2.

### Example 6

### Dopamine does not Affect Foxp3 Expression by Treg

A gene encoding the Foxp3 protein was recently found to be associated with Treg (Hori et al., 2003; Ramsdell, 2003). We therefore examined whether the dopamine-induced reduction of Treg activity alters the expression of this gene. mRNA isolated from Treg that were activated for 24 h, exposed for 2 h to dopamine, and maintained in culture for a further 30 min or 24 h was analyzed for Foxp3 expression. Foxp3, as expected, was detected in Treg, but no significant change in its expression was observed after Treg were exposed to dopamine for 30 min (FIG. 2m) or 24 h (data not shown).

### Example 7

### ERK1/2 is Deactivated by Dopamine in Treg

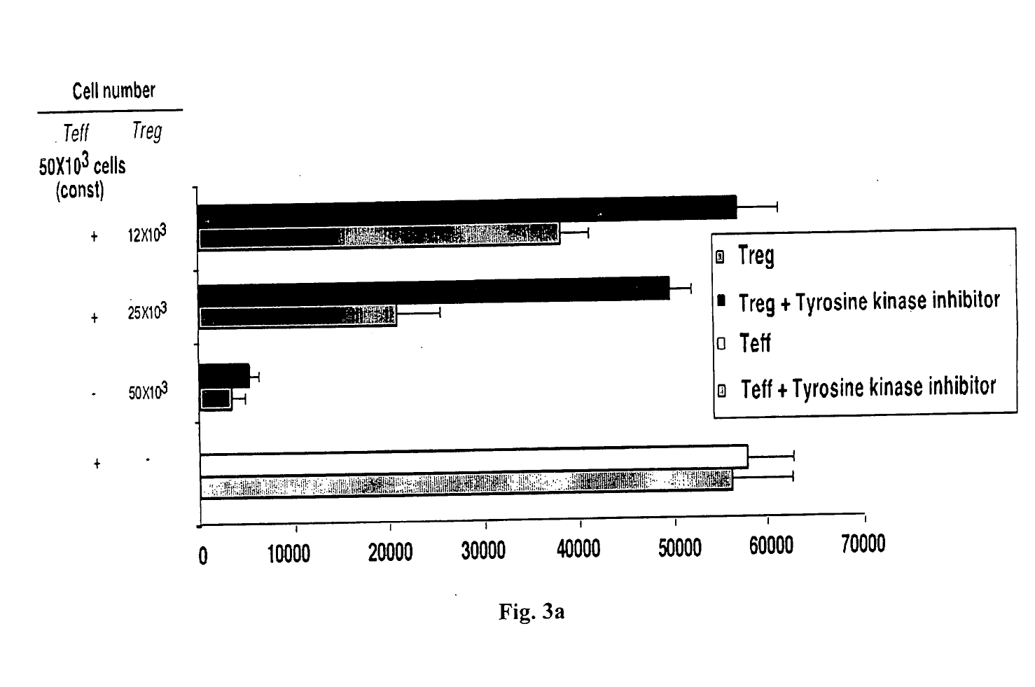

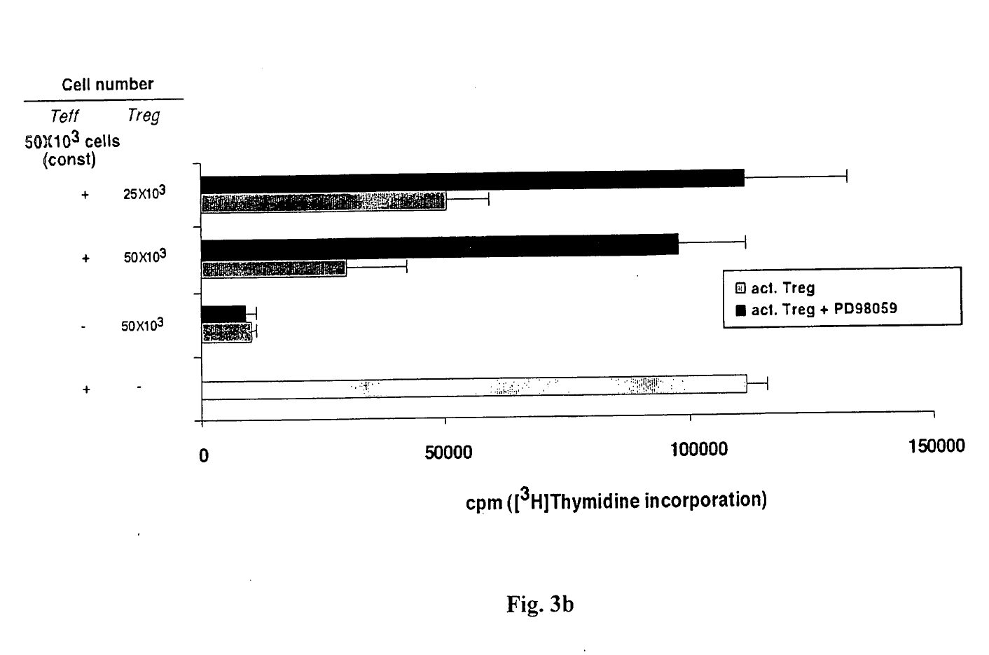

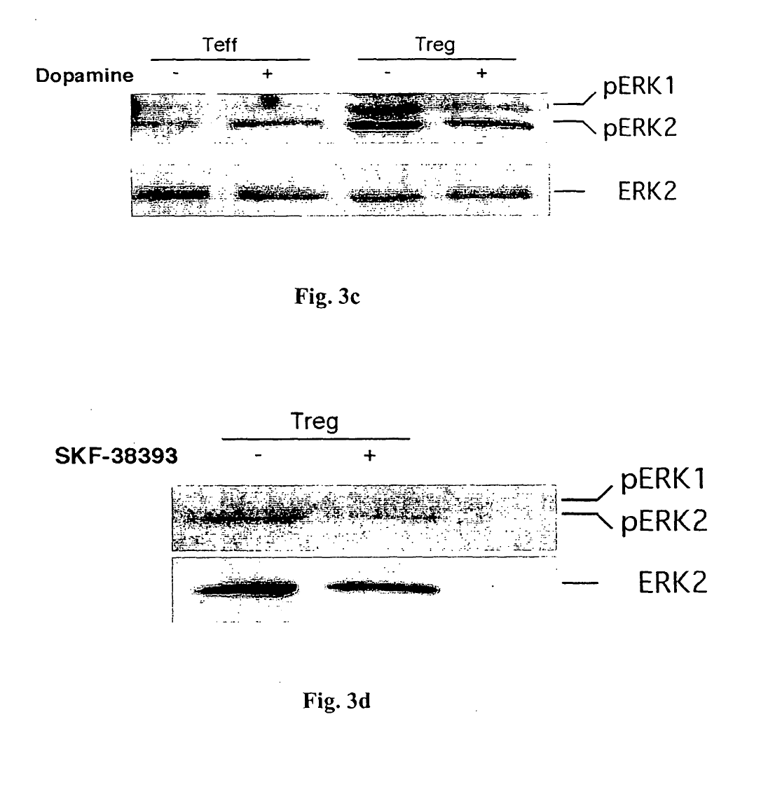

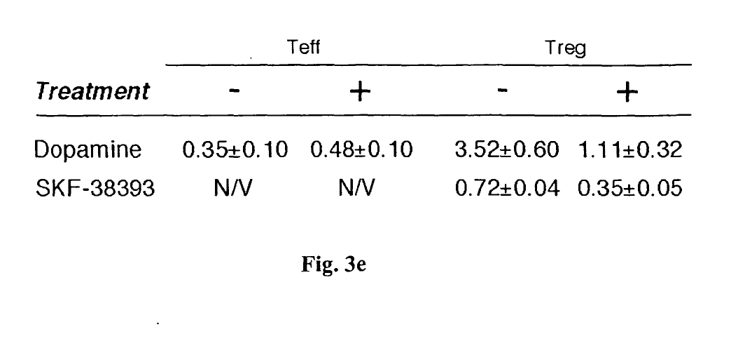

The finding that dopamine down-regulated Treg activity via D1-type but not D2-type receptors, taken together with the recent report that the ERK pathway can be activated by D1-R-dependent signaling (Takeuchi and Fukunaga, 2003), led us to suspect that the down-regulatory effect of dopamine on the suppressive activity of Treg might be exerted via the ERK pathway. To examine this possibility we first treated Treg with the protein tyrosine kinase inhibitor genistein (4′,5,7-trihydroxy isoflavone), which inhibits ERK and MEK activation (Mocsai et al., 2000). This treatment blocked the suppressive activity of Treg on Teff (FIG. 3a). It is interesting to note that, in the presence of genistein, Treg not only lost their suppressive activity but even underwent proliferation themselves. Genistein at the same concentration had no effect on the proliferation of Teff (FIG. 3a).

In light of these results, we also examined whether Treg activity is affected by PD98059, a specific MEK inhibitor that blocks the ERK1/2 signaling pathway (Sharp et al., 1997). PD98059 significantly reduced the suppressive activity of Treg relative to that of control activated Treg (FIG. 3b).

The above findings prompted us to examine the state of ERK phosphorylation in activated Treg and in Treg that were activated in the presence of dopamine. Treg were activated for 20 min in the presence or absence of dopamine (10⁻⁵ M), and were then subjected to intracellular phosphoprotein staining (Perez and Nolan 2002) and analyzed by flow cytometry. Significantly less phosphorylated ERK was detected in Treg that were activated in the presence of dopamine than in activated Treg without dopamine (data not shown). As a measure of the background nonspecific staining of the phosphorylated ERK we used a specific peptide of phospho-ERK, which competes for binding of the antibody. The intracellular staining procedure for phospho-ERK detection by FACS was validated by the use of Teff incubated with 20 μM phorbol 12-myristate 13-acetate (PMA), known to stimulate the activity of protein kinase C (PKC) (data not shown). To further substantiate these findings, we performed Western blot analysis of phospho-ERK1/2 expression in lysates of Treg and Teff after the cells had been activated with anti-CD3 and anti-CD28 for 20 min in the presence or absence of dopamine. Significantly more phosphorylated ERK1/2 was detected in activated Treg than in activated Teff. Moreover, phospho-ERK1/2 was found to be down-regulated in Treg that had been activated in the presence of dopamine (FIG. 3c). ERK1/2 phosphorylation in Treg was also reduced by the specific D1-type receptor agonist SKF-38393 (FIG. 3d). Results of quantitative analysis of the phospho-bands are shown in FIG. 3e.

### Example 8

### Dopamine Alters the Adhesive and Migratory Properties of Treg

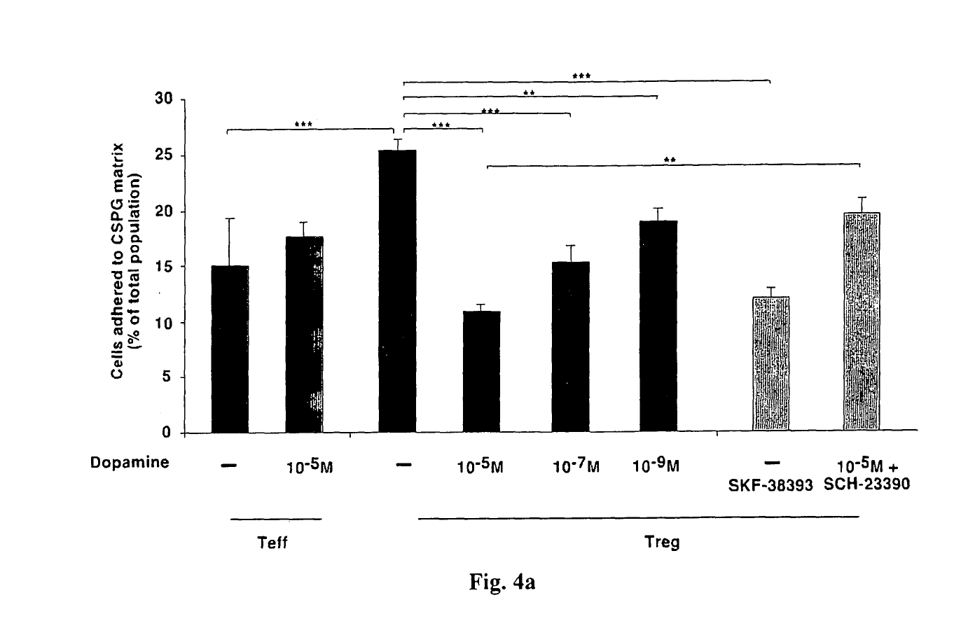

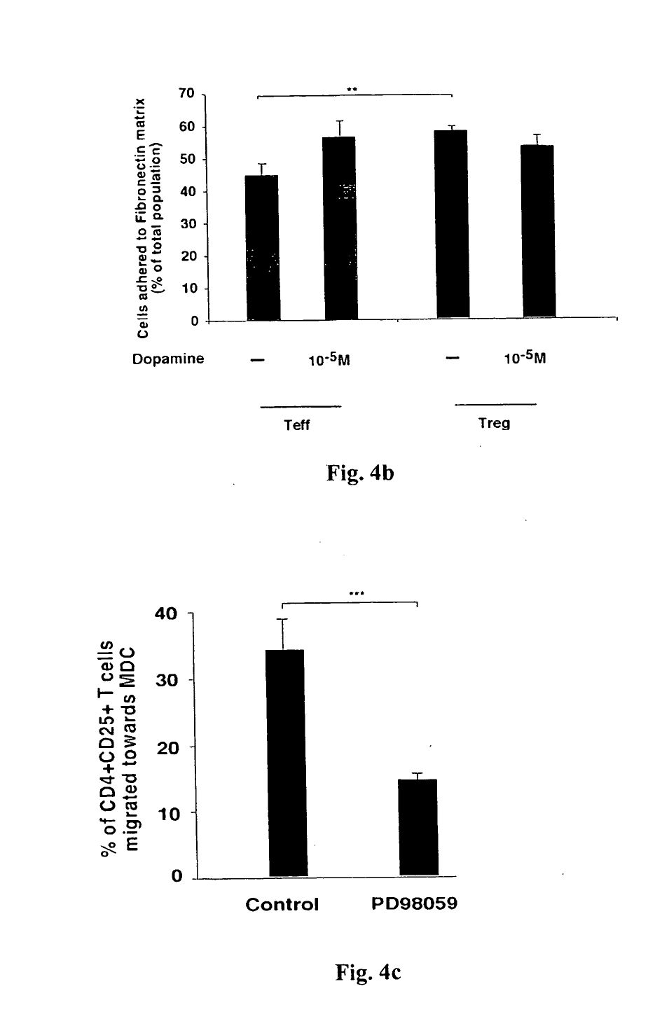

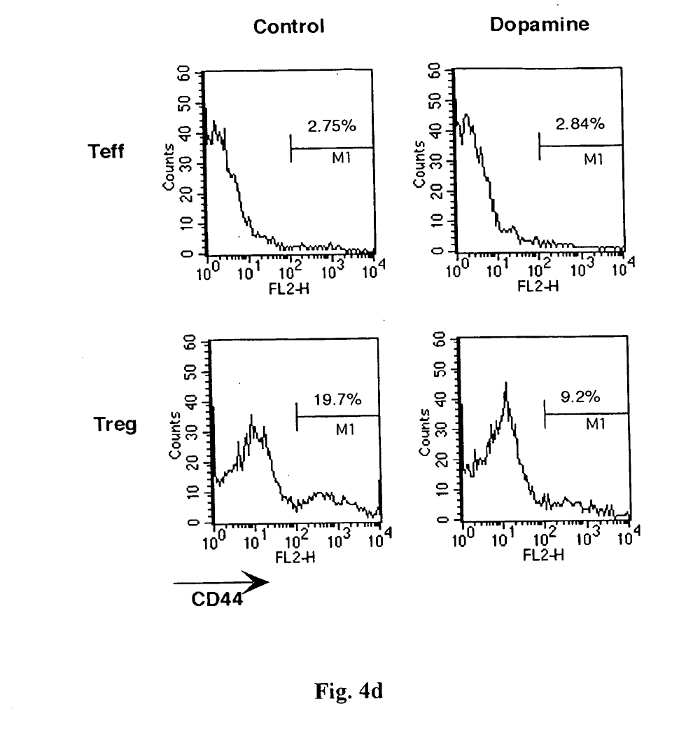

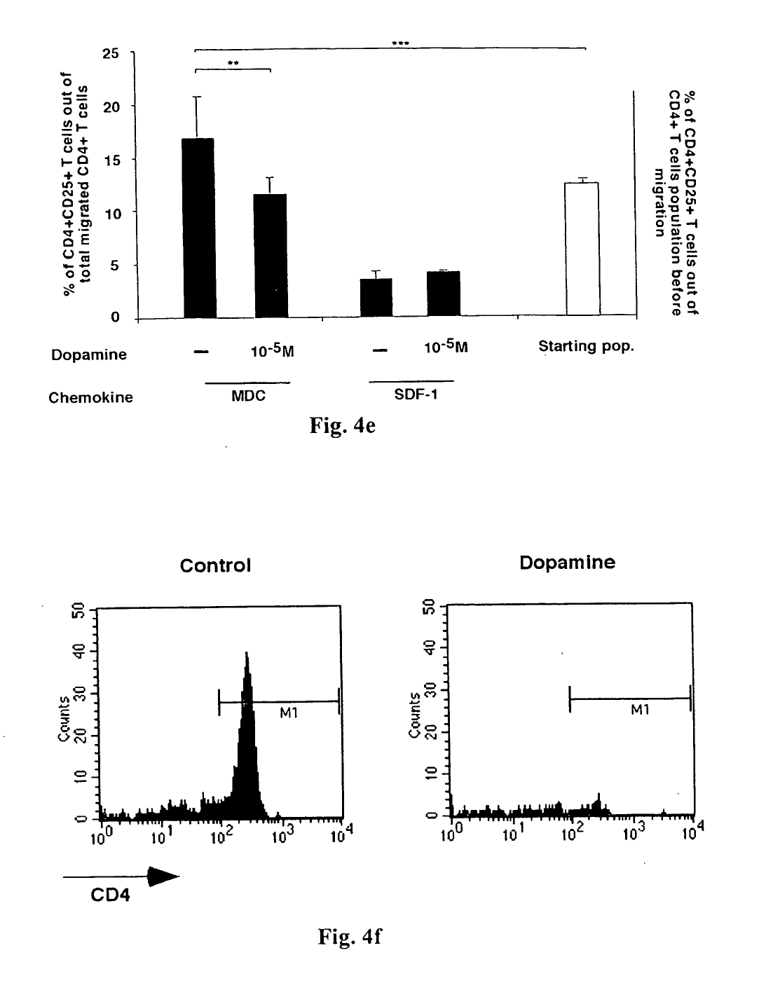

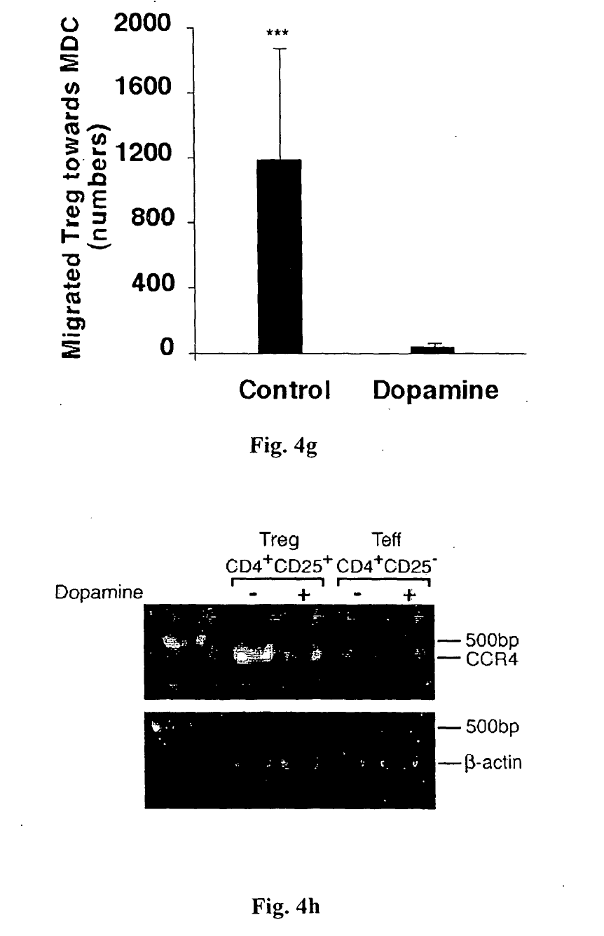

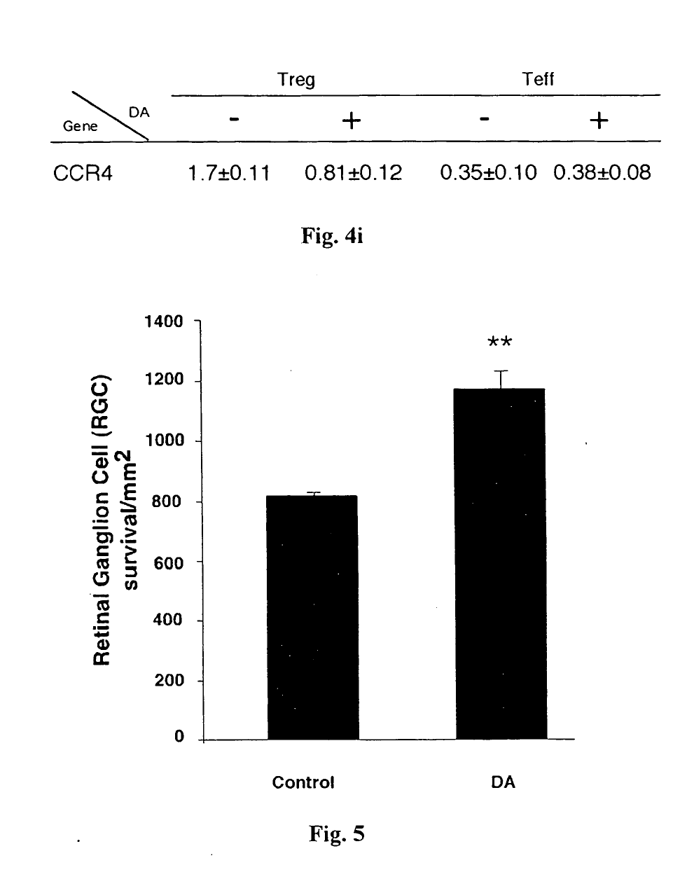

One of the main features of T cells is their ability to migrate to tissues in need of rescue or repair [such as diseased or damaged CNS (Hickey, 1999; Flugel et al., 2001). We therefore considered the possibility that dopamine reduces not only the suppressive activity but also the migratory ability of Treg. Since T cell migration and adhesion have been linked to ERK activation (Tanimura et al., 2003), this assumption appeared even more feasible in light of the above observation that dopamine reduced ERK activation in Treg. We therefore incubated Treg with dopamine for 2 h and then examined their adhesion to chondroitin sulfate proteoglycans (CSPG), extracellular matrix proteins often associated with injured tissues (Jones et al., 2003). The ability of Treg to adhere to CSPG was significantly greater than that of Teff (FIG. 4a), and was significantly decreased, in a concentration-dependent manner (10⁻⁹-10⁻⁵ M), by dopamine (FIG. 4a). The dopamine effect on Treg could be mimicked by the D1-type specific agonist SKF-38393 and inhibited by the D1-type antagonist SCH-23390. Dopamine had only a slight, nonsignificant effect on the adhesion of Teff to CSPG (FIG. 4a). The ability of Treg to adhere to fibronectin was greater than that of Teff (FIG. 4b). Exposure to dopamine resulted in no effect on adhesion of Treg to fibronectin and a slight increase in the adhesion of Teff (FIG. 4b).

To verify that the effect of dopamine on adhesion of Treg is exerted through the ERK1/2 pathway, we incubated Treg with the ERK1/2 signaling pathway inhibitor PD98059 before carrying out the adhesion assay. PD98059 significantly reduced the ability of Treg to adhere to CSPG (FIG. 4c). Since interaction of T cells with CSPG is mediated in part by the CD44 receptor (Henke et al., 1996), and in light of the known dependence of CD44 expression on the ERK signaling pathway, it was conceivable that dopamine might affect the expression of CD44 in Treg. To examine this possibility, we assayed CD44 immunoreactivity in Treg and Teff that had been activated with anti-CD3 and anti-CD28 antibodies for 24 h and then incubated for 2 h with or without dopamine. In the absence of dopamine, CD44 immunoreactivity was significantly stronger in Treg than in Teff. Dopamine decreased CD44 immunoreactivity in Treg but not in Teff (FIG. 4d). Other adhesion-molecule receptors that we tested, such as LFA-1, I-CAM, and V-CAM (Lee and Benveniste, 1999), did not show any dopamine-related changes in Treg (data not shown).

Migration of Treg in humans is dependent on the chemokine receptors CCR-4 and CCR-8, which are abundantly present on Treg (Sebastiani et al., 2001). We therefore examined whether exposure to dopamine would also affect Treg migration. For this experiment we used a normal population of CD4⁺ T cells, of which Treg (CD4⁺CD25⁺) accounts for approximately 11% (FIG. 4e). Of the CD4⁺, cells that migrated towards CCL22 (MDC; a chemokine for CCR-4), 17% were Treg (CD4⁺CD25⁺), pointing to the greater migratory ability of Treg than of Teff towards MDC. However, after exposure of the CD4⁺ cell population to dopamine, migrating Treg accounted for approximately 10% (the same as their percentage in the total CD4⁺ population at the start of the experiment), suggesting that after their exposure to dopamine Treg lost their preference for migration towards MDC.

We also examined the migration of a mixed T cell population towards SDF-1. Migration of Teff towards SDF-1 was significantly greater than that of Treg (post-migration percentage of Treg in the total CD4⁺ population was less than 4%), and dopamine did not alter this pattern (FIG. 4e). To examine the direct effect of dopamine on the migration of Treg, we assayed the effect of dopamine on the migration of purified Treg towards MDC. The migratory Treg were stained for CD4 to ensure that cell debris and aggregates would not be counted among them. Dopamine almost completely abolished Treg migration (FIGS. 4f, 4g), but had no effect on the migration of Teff (data not shown).

In an attempt to link the changes in migration to specific receptors, we examined the expression of mRNA for CCR-4, CCR-8, and CXCR-4. Before the cells were exposed to dopamine, their CCR-4 expression—as expected from previous findings in human Treg (Sebastiani et al., 2001, 2002)—was significantly higher in Treg than in Teff, but upon exposure to dopamine the expression of CCR-4 in Treg was decreased (FIGS. 4h, 4i). The expression of mRNA encoding for CXCR-4 and CCR-8 did not change in Treg after these cells were exposed to dopamine (data not shown).

### Example 9

### Exogenous Dopamine Increases the Ability to Fight Off Neurodegeneration

A previous study by our group showed that injection of activated Treg into mice (Balb/c) immediately after CNS injury significantly inhibits the spontaneous neuroprotective response, with the result that fewer neurons survive the consequences of the insult (Kipnis et al., 2002a). In the same study we showed that depletion of Treg increases the ability to withstand the insult. The present observation that Treg and Teff respond differentially to dopamine prompted us to examine the effect of dopamine on the ability to withstand neurotoxic conditions in vivo.

9a. Dopamine Affords Neuroprotection after CNS Injury

Reasoning that systemic injection of dopamine after a CNS insult would be expected to improve recovery after a mechanical CNS injury, we subjected two groups (n=12 in each group) of BALB/c mice to a severe optic nerve crush injury (a known model of secondary neuronal degeneration) and immediately thereafter injected the mice in one group with dopamine (0.4 mg/kg) and those in the other group with PBS. Two weeks later their retinas were excised and neuronal survival assessed. Significantly more viable neurons (1110±56/mm², mean±SD) were found in the retinas of dopamine-injected mice than in the retinas of vehicle-treated mice (789±23; FIG. 5). Thus systemic injection of dopamine led to an increased ability to cope with consequences of optic nerve injury.

9b. Dopamine Protects from Neuronal Toxicity Induced by Glutamate

To assess whether the beneficial effect of systemic dopamine is a general phenomenon, rather than unique to a single animal model, we used a model of neuronal toxicity induced by glutamate, a common player in many neurodegenerative conditions (Katayama et al., 1990; Xiong et al., 2003; Jiang et al., 2001). Injection of glutamate into the eyes of adult mice causes retinal ganglion cell death that is measurable 1 week after the injection (Mizrahi et al., 2002; Schori et al., 2001a; Katayama et al., 1990). We injected Balb/c mice intraperitoneally (i.p.) with the dopamine, or its specific D1-type agonist SKF-38393 (3.3 mg/kg), or its specific D1-type antagonist SCH-23390 (3 mg/kg), immediately after their exposure to glutamate toxicity. We also injected SCID Balb/c mice with SKF-38393 (3.3 mg/kg) immediately after glutamate intoxication. Since the glutamate toxicity model, irrespective of the treatment approach, leaves only a narrow therapeutic window, the number of protected neurons is expressed here as a percentage of the total number of neurons amenable to protection. A single systemic injection of dopamine (0.4 mg/kg) or its D1-type agonist given immediately after intraocular injection of a toxic dose of glutamate increased neuronal survival by 18±2.5 or 19±3.2%, respectively, relative to that in glutamate-injected controls treated with PBS (Table 1). Injection of the same agonist to SCID mice resulted in no effect, thus supporting the assumption that systemic dopamine benefit CNS neurons via the peripheral immune system. As a corollary, injection of the D1-type antagonist resulted in a decrease in neuronal survival (11±1.5%, p<0.01; Table 1) relative to that in PBS-injected mice. The above results suggested that dopamine might be one of the endogenous signals initiating the cascade that leads to spontaneous T cell-dependent neuroprotection. Accordingly, a single injection of mice with a D1-type antagonist would be expected to exacerbate neuronal survival, as it would compete with the endogenous dopamine for reduction of the suppressive activity of Treg after an injury.

TABLE 1
Neuronal survival following glutamate intoxication in mice
injected with dopamine or its type-1 receptor agonist and antagonist.

| | Treatment | | |
|---|---|---|---|
| Mice | Dopamine | SKF-38393 | SCH-23390 |
| Wild type | 18 ± 2.5*** | 19 ± 3.2** | −11 ± 1.5** |
| SCID | NT | 3 ± 1.8 (ns) | NT |

Immediately after glutamate intoxication mice were systemically injected with the indicated drugs. Neuronal survival was determined ten days later (see Materials and Methods). The results are expressed by changes (in percentage) in neuronal survival in treated mice relative to untreated mice. Each value represents a mean±SEM of a group of at least 5 animals, and each experiment was performed at least twice, independently. Asterisks (***, P<0.001; **, P<0.01) indicate statistical significance of the presented data from a single experiment using a Student's T-test statistical analysis. (NT—not tested; ns—no statistical significance).

### Example 10

### Exposure of Treg to Dopamine In Vitro Reduces their Suppressive Activity In Vivo

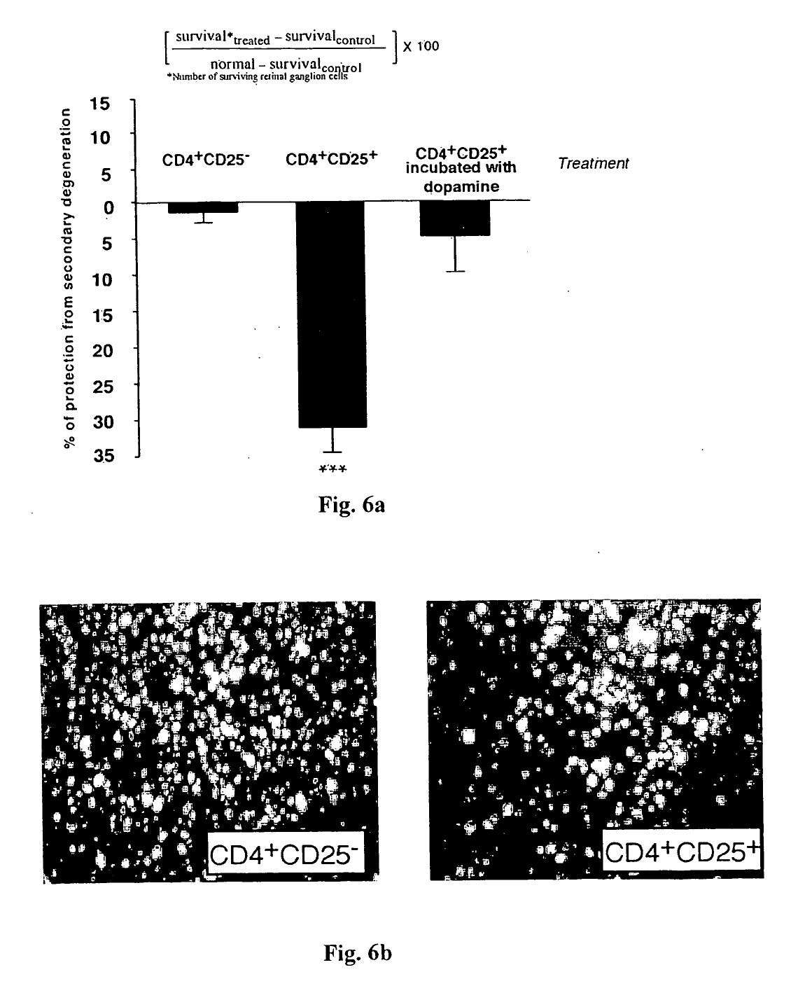

To unequivocally show the direct effect of dopamine on Treg activity in vivo, we examined whether direct exposure of Treg to dopamine can reduce their suppressive activity in a model of neuronal survival. Systemic injection of Treg after glutamate intoxication significantly reduced the ability of the mice to withstand the glutamate toxicity and resulted in a 25% increase in neuronal death. We further found, however, that incubation of Treg with dopamine prior to their systemic injection into mice abolished their suppressive effect, indicated by the lack of change in the number of surviving neurons. No effect on neuronal survival after glutamate intoxication could be detected in control mice injected with Teff (FIG. 6a). FIG. 6b shows representative micrographs of fields from retinas excised from mice that were exposed to intravitreally injected glutamate and then injected with either CD4⁺CD25⁺ or CD4⁺CD25⁻.

### Example 11

### The D1-R Agonist SKF-38393 Protects Mice from Glutamate Toxicity

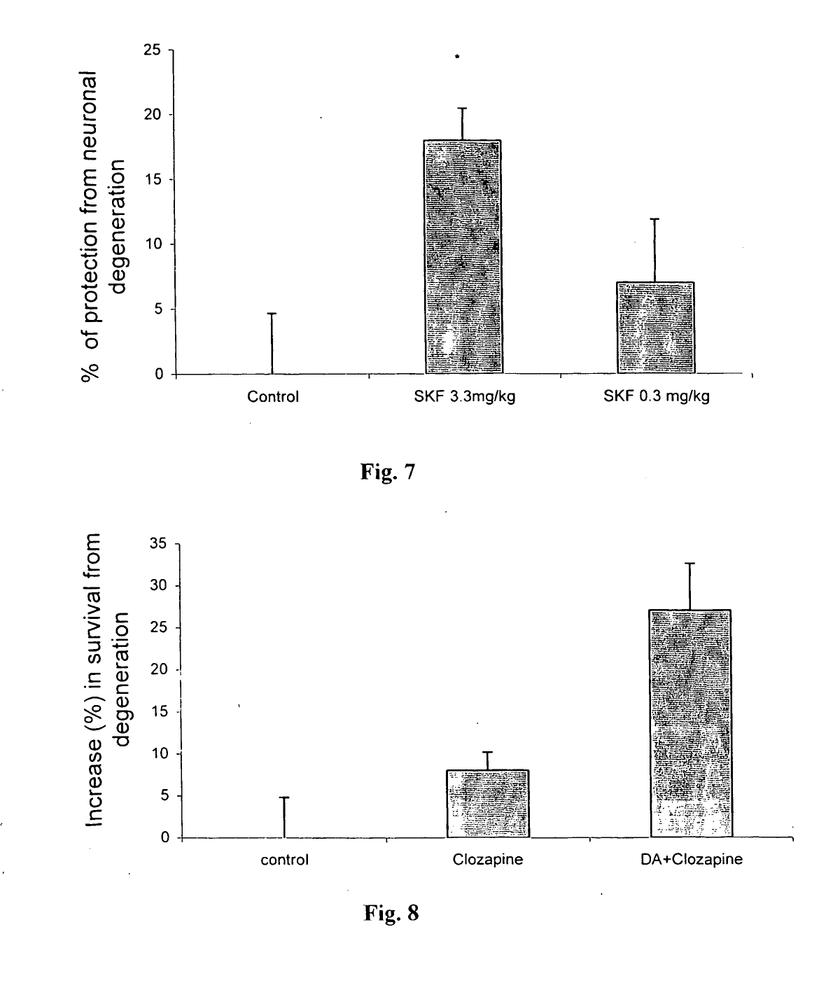

Mice were injected intra-ocular with a toxic dose of glutamate followed by an immediate injection i.v of the D1-R family agonist SKF-38393. Retinas were excised 7 days afterwards and survived neurons were counted. The results are depicted in FIG. 7. Mice injected with 3.3 mg/kg of SKF-38393 showed significant increase in neuronal survival compared to vehicle-injected mice. Injection of a lower dose of SKF-38393 (0.33 mg/kg) showed a neuroprotective trend, however not significant.

### Example 12

### Clozapine Alone or with Dopamine Protects Mice from Glutamate Toxicity

Mice were injected with a toxic dose of glutamate into the eyes followed by an immediate injection i.v of the D2-R family antagonist clozapine (5 mg/kg) or with clozapine in combination with dopamine (a mixture of 0.4 mg/kg of dopamine with 0.6 mg/kg of clozapine). Retinas were excised 7 days afterwards and survived neurons were counted. The results are depicted in FIG. 8. Mice injected with clozapine alone showed a significant increase in neuronal survival compared to vehicle-injected mice. Moreover, mice injected with clozapine in combination with dopamine showed even higher neuronal survival.

### Example 13

### Effect of Dopamine and Dopamine D1-R Agonist in Cancer

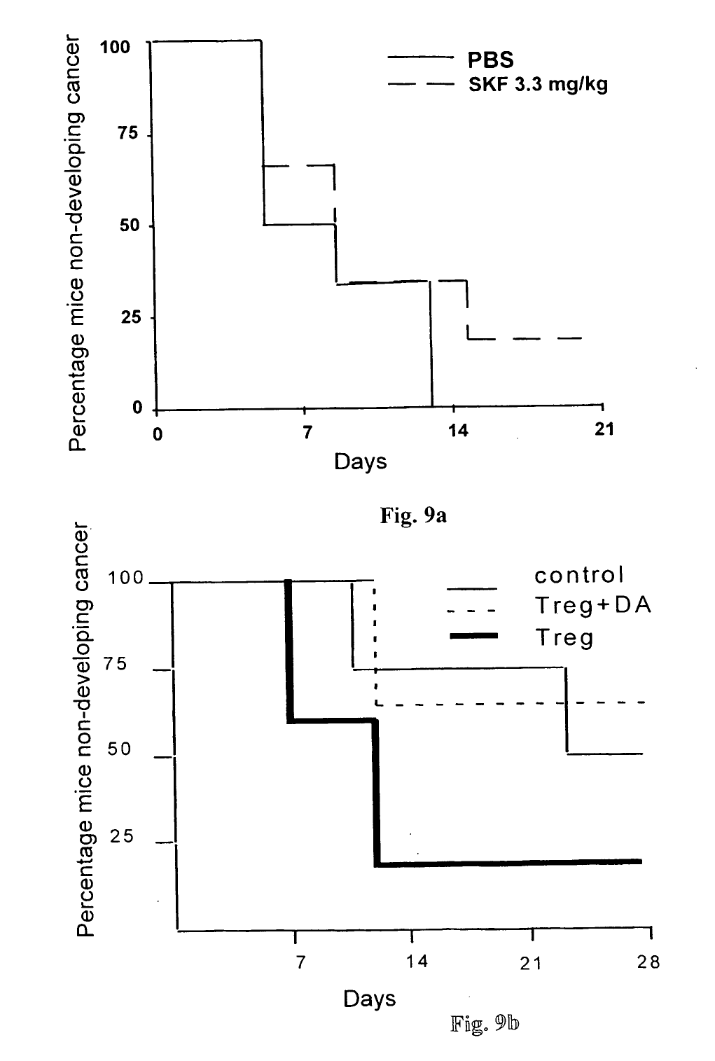

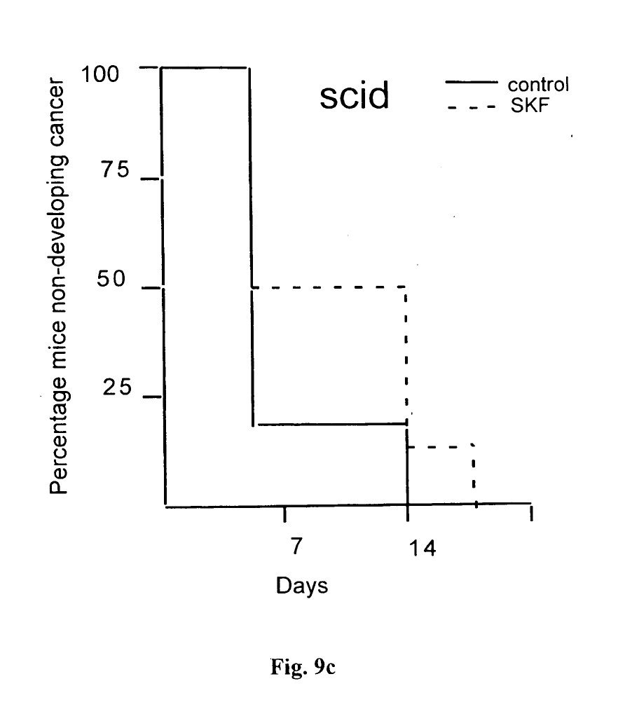

To verify the effect of dopamine and its related compounds on tumor growth, C57BL mice were injected with the specific D1-R agonist SKF-38393 (3.3 mg/kg) immediately after inoculation of mouse solid melanoma tumor cells M2R. The experimental group (n=10) received a single injection of SKF-38393, and a control group (n=11) was injected with PBS. Animals in control group started to develop tumor at day 6 post inoculation and after 13 days all animals developed tumors. Animals, which were injected with SKF-38393 after tumorigenic cell inoculation the tumor development was significantly attenuated (FIG. 9a).

In another experiment, mice (n=10) were inoculated with solid tumor cells M2R, and received an immediate injection of Treg (2×10⁶ cells) with and without exposure to dopamine (10⁻⁵M). Control animals (n=10) received an injection of vehicle (PBS) following cancer cells inoculation. Animals injected with Treg showed increased incidence and course of tumor development than control animals (PBS injected following M2R inoculation). Mice injected with Treg exposed to dopamine did not differ from control mice, suggesting that the Treg had lost their suppressive activity as a result of their encounter with dopamine (FIG. 9b).

To further isolate the effect of dopamine on the immune system, C57BL mice (n=10) and SCID mice (n=8) were injected with SKF-38393 (3.3 mg/kg) immediately after injecting them with solid tumor cells M2R (FIG. 9c). No effect of SKF was observed in the absence of endogenous immune system, indicating that dopamine works through immune system.

### Discussion

The results above show that dopamine reduces the suppressive and trafficking activities of Treg through a family of type-1 dopamine receptors (D1-R and D5-R, found here to be abundantly expressed by Treg) via the ERK signaling pathway. The physiological and pharmacological effects of dopamine, as a compound capable of down-regulating Treg activity needed for fighting off neurodegeneration by T cell-dependent mechanism, is shown in models of CNS insult and cancer.

Glutamate is a common mediator of CNS neurodegenerative conditions

(Urushitani et al., 1998; Rothstein, 1995-96; Newcomer et al., 1999; Lasley and Gilbert, 1996; Gunne and Andren, 1993). Recent studies strongly suggest that the ability to withstand CNS insults, including glutamate toxicity, is T-cell dependent and is amenable to boosting by self-antigens residing in the site of damage (Moalem et al., 1999; Mizrahi et al., 2002; Yoles et al., 2001; Kipnis et al., 2001; Schori et al., 2001; Hauben et al., 2000; Wekerle, 2000).

An alternative way to achieve beneficial enhancement of the autoimmune response against the self-antigens needed for protection and repair after a CNS injury or for fighting off tumors is by eliminating the normally suppressive effect of Treg (Kipnis et al., 2002; Sakaguchi et al., 2001; Shimizu et al., 1999). Physiological compound(s) that control Treg activity on a daily basis probably underlie the mechanisms whereby the body overcomes commonly occurring adverse conditions, which in most cases resolve without development of tumors or neuronal degeneration.

The results of the present invention indicate that one such physiological compound is dopamine. In this context it is important to note that transient changes in dopamine levels in mesolimbic brain areas in rats, associated with neuronal activity, can reach concentration as high as 600 nM (Gonon, 1997; Floresco et al., 2003; Wightman and Robinson, 2002). It was also reported that blood levels of dopamine are elevated in patients with certain types of tumors (Saha et al., 2001).

According to the present invention, dopamine reduced Treg activity, and this was correlated with a decrease in ERK1/2 activation. In line with this observed correlation was the finding that adhesive and migratory abilities of Treg (Pozzi et al., 2003; Takeuchi and Fukunaga, 2003; Tanimura et al., 2003; Lohse et al., 1996; Schneider et al., 2002; Yi et al., 2002), were reduced by dopamine via the ERK pathway.

Treg might exert their suppressive activity on Teff (autoimmune T cells) either in the lymphoid organs or at the site of the threat (degeneration or tumor growth). Mediation of the suppressive activity of Treg has been attributed partially to IL-10 and CTLA-4, whereas their migration and adhesion have been attributed to the specific repertoire of chemokine receptors and adhesion molecules that they express (Kohm et al., 2002; Sebastiani et al., 2001). Reduction of the suppressive activity of Treg was correlated with a decrease in their IL-10 production and CTLA-4 expression, which might participate in the cytokine-mediated and cell-cell mediated suppression by Treg, respectively. Moreover, Treg express relatively large amounts of the CD44 receptor (needed for their adhesion to CSPG) and the chemokine receptor CCR-4 (needed for their migratory ability). Exposure of Treg to dopamine resulted in a decrease in both their adhesion to CSPG and their migration towards MDC, in correlation with their diminished expression of CD44 and CCR-4, respectively.

The ability of dopamine to affect Treg and Teff differently, as observed in the present invention, is probably related to both the unique nature of dopamine receptors and the nature of their expression on these two T-cell populations. We found that significantly more D1-R and D5-R are expressed by Treg than by Teff. The marked difference in D1-R and D5-R expression, which is hardly detectable on Teff or any other immune cells (Ricci et al., 1997), makes the D1-type receptor family a likely candidate for the dialog of dopamine with Treg, leading—via the ERK pathway—to reduction of the suppressive activity of Treg. It is interesting to note that D2-R, which antagonizes D1-R, activates ERK (Pozzi et al., 2003).

We found that the effect of dopamine on the suppressive activity of Treg was weak compared with the effect of a protein tyrosine kinase inhibitor such as genistein (Mocsai et al., 2000) or the ERK1/2 signaling pathway inhibitor PD98059, indicating that dopamine is a suitable candidate as a physiological immunomodulator mainly in the context of autoimmune activity.

Treg exist in a state of anergy, neither proliferating in response to mitogenic stimuli nor producing IL-2. Although dopamine down-regulated the suppressive activity of Treg, it did not reverse the anergic state of these T cells with respect to proliferation or IL-2 production, supporting the contention that dopamine induces changes in the activity rather than in the phenotype of Treg. Unlike dopamine, genistein not only blocked the activity of Treg but also triggered their proliferation, suggesting that under extreme conditions the phenotype of Treg might change.

The in-vivo relevance of the effect of dopamine on the suppressive activity of Treg was demonstrated according to the present invention in the experimental paradigms of glutamate intoxication in the mouse eye and mouse optic nerve mechanical crush injury. After glutamate intoxication, passive transfer of Treg suppressed the ability to resist neurodegeneration, as indicated by an increased loss of neurons. Incubation of Treg with dopamine prior to their transfer wiped out their suppressive effect on neuronal survival. The loss of Treg activity in vivo might reflect the effect of dopamine both on homing of Treg to the damaged site and on their suppression. To further investigate the potential activity of dopamine as an immunomodulator, we injected it systemically. Significantly more neurons survived a neurotoxic insult in mice injected with dopamine or its D1-type agonist than in PBS-injected controls. A similar effect was obtained when the mouse received a systemic injection of dopamine after an optic nerve crush injury.

It is important to note that dopamine, when injected systemically, does not cross the blood-brain barrier. The observed lack of effect of the D1-type agonist SKF-38393 in nude mice (which are devoid of mature T cells) substantiated our conclusion that the effect of peripheral dopamine on neuronal survival is exerted via the immune system and not directly on neural tissue. The potential ability of endogenous dopamine to operate spontaneously in vivo as a stress-related signal, emitted by the CNS to the peripheral immune system after a CNS insult, was demonstrated in mice injected with SCH-23390 immediately after glutamate intoxication. Mice injected with this D1-type antagonist showed a slight (11%) but significant decrease in neuronal survival. The weak effect of SCH-23390 on neuronal survival appears to be attributable, at least in part, to the nature of the experimental model. Thus, under the experimental conditions of this study, nude mice lost approximately 30% of their neurons relative to the wild type (Kipnis et al., 2002; Schori et al., 2002). The 10% decrease observed in the wild-type mice resulting from manipulation of Treg activity therefore represents more than 30% of the maximal possible effect. It is also possible that dopamine is a member of a family of physiological compounds capable of controlling Treg activity after a CNS insult.

Previous studies have documented the effect of dopamine on T cell adhesion (Levite et al., 2001), on activation (Ilani et al., 2001), and on T-lymphocyte suppression of IgG production by peripheral blood mononuclear cells (Kirtland et al., 1980). No attempt was made in any of those studies to attribute the dopamine effect to subpopulations of CD4+ T cells. Subsequent studies showed that dopamine exerts its effect only on activated T cells (Ilani et al., 2001). Our results suggest that dopamine has a direct and preferential effect on Treg in initiating the immune response but will not circumvent the need for the two signals known to be needed for eliciting a T cell response [antigen recognition by T cells on class II major histocompatibility complex (MHC-II) proteins and co-stimulatory molecules (Bretscher and Cohn, 1970)]. It was recently suggested that in the presence of strong immunogens, Teff, with the aid of APCs, can overcome the suppression imposed by Treg (Pasare and Medzhitov, 2003). This mechanism is not likely to operate in response to self-antigens, possibly because the self-antigens are neither present in sufficient amounts nor sufficiently potent to induce the needed response.

In light of the observed effect of dopamine on Treg in the present invention, the uncontrolled presence of dopamine known to occur in patients with mental disorders (such as schizophrenia) might explain the high incidence of aberrant immune activity in these patients (Muller et al., 2000). It is also interesting to note the relatively low incidence of cancer development observed in patients with schizophrenia (Teunis et al., 2002), in whom dopaminergic activity is known to be pronounced. This apparently unexplained phenomenon could be interpreted in light of the present finding of the dopamine effect on Treg, as well as the known participation of autoimmune T cells in fighting off cancer (Dummer et al., 2002).

The observed correlation between the state of ERK activation and the activity of Treg opens the way, via dopamine or its related compounds, to novel therapeutic strategies for fine-tuning of Treg activity, and hence for fighting off conditions in which Treg activity needs to be weakened (such as neuronal degeneration and cancer) or strengthened (autoimmune diseases).

The findings of the present invention shed light on the physiological mechanisms controlling Treg and opens the way to novel therapeutic strategies by using dopamine as well as dopamine agonists or antagonists as candidates for therapy against cancer and neurodegenerative diseases, by down-regulating the suppressive activity of Treg, or for treatment of autoimmune diseases and prevention of graft rejection by up-regulating the suppressive activity of Treg.

### REFERENCES

Basu S, Nagy J A, Pal S, Vasile E, Eckelhoefer I A, Bliss V S, Manseau E J, Dasgupta P S, Dvorak H F, Mukhopadhyay D. 2001. The neurotransmitter dopamine inhibits angiogenesis induced by vascular permeability factor/vascular endothelial growth factor. Nat Med 7:569-574.
Bretscher, P., and M. Cohn. 1970. A theory of self-nonself discrimination. Science 169:1042-1049.
Colao A, Di Sarno A, Landi M L, Cirillo S, Sarnacchiaro F, Facciolli G, Pivonello R, Cataldi M, Merola B, Annunziato L, Lombardi G. 1997. Long-term and low-dose treatment with cabergoline induces macroprolactinoma shrinkage. J Clin Endocrinol Metab 82:3574-3579.
Dummer, W., A G. Niethammer, R. Baccala, B. R. Lawson, N. Wagner, R. A. Reisfeld, and A. N. Theofilopoulos. 2002. T cell homeostatic proliferation elicits effective antitumor autoimmunity. J Clin Invest 110:185-192
Durif F, Vidailhet M, Assal F, Roche C, Bonnet A M, Agid Y. 1997. Low-dose clozapine improves dyskinesias in Parkinson's disease. Neurology 48:658-662.
Edgar, V. A., G. A. Cremaschi, L. Sterin-Borda, and A. M. Genaro. 2002. Altered expression of autonomic neurotransmitter receptors and proliferative responses in lymphocytes from a chronic mild stress model of depression: effects of fluoxetine. Brain Behav Immun 16:333-350.
Fancellu R, Armentero M T, Nappi G, Blandini F. 2003. Neuroprotective effects mediated by dopamine receptor agonists against malonate-induced lesion in the rat striatum. Neurol Sci 24:180-181.
Floresco, S. B., A. R. West, B. Ash, H. Moore, and A. A. Grace. 2003. Afferent modulation of dopamine neuron firing differentially regulates tonic and phasic dopamine transmission. Nat Neurosci 6:968-973.
Flugel, A., T. Berkowicz, T. Ritter, M. Labeur, D. E. Jenne, Z. Li, J. W. Ellwart, M. Willem, H. Lassmann, and H. Wekerle. 2001. Migratory activity and functional changes of green fluorescent effector cells before and during experimental autoimmune encephalomyelitis. Immunity 14:547-560.
Gonon, F. 1997. Prolonged and extrasynaptic excitatory action of dopamine mediated by D1 receptors in the rat striatum in vivo. J Neurosci 17:5972-5978.
Goth M I, Hubina E, Raptis S, Nagy G M, Toth B E. 2003. Physiological and pathological angiogenesis in the endocrine system. Microsc Res Tech 60:98-106.
Gunne, L. M., and P. E. Andren. 1993. An animal model for coexisting tardive dyskinesia and tardive parkinsonism: a glutamate hypothesis for tardive dyskinesia. Clin Neuropharmacol 16:90-95.
Henke, C. A., U. Roongta, D. J. Mickelson, J. R. Knutson, and J. B. McCarthy. 1996. CD44-related chondroitin sulfate proteoglycan, a cell surface receptor implicated with tumor cell invasion, mediates endothelial cell migration on fibrinogen and invasion into a fibrin matrix. J Clin Invest 97:2541-2552.
Hickey, W. F. 1999. Leukocyte traffic in the central nervous system: the participants and their roles. Semin Immunol 11:125-137.
gHori, S., T. Nomura, and S. Sakaguchi. 2003. Control of regulatory T cell development by the transcription factor Foxp3. Science 299:1057-1061.
Ilani, T., D. Ben-Shachar, R. D. Strous, M. Mazor, A. Sheinkman, M. Kotler, and S. Fuchs. 2001. A peripheral marker for schizophrenia: Increased levels of D3 dopamine receptor mRNA in blood lymphocytes. Proc Natl Acad Sci USA 98:625-628.
Im, S. H., D. Barchan, P. K. Maiti, S. Fuchs, and M. C. Souroujon. 2001. Blockade of CD40 ligand suppresses chronic experimental myasthenia gravis by down-regulation of Th1 differentiation and up-regulation of CTLA-4. J Immunol 166:6893-6898.
Jones, L. L., R. U. Margolis, and M. H. Tuszynski. 2003. The chondroitin sulfate proteoglycans neurocan, brevican, phosphacan, and versican are differentially regulated following spinal cord injury. Exp Neurol 182:399-411.
Jiang, Z. G., C. Piggee, M. P. Heyes, C. Murphy, B. Quearry, M. Bauer, J. Zheng, H. E. Gendelman, and S. P. Markey. 2001. Glutamate is a mediator of neurotoxicity in secretions of activated HIV-1-infected macrophages. J Neuroimmunol 117:97-107.
Katayama, Y., D. P. Becker, T. Tamura, and D. A. Hovda. 1990. Massive increases in extracellular potassium and the indiscriminate release of glutamate following concussive brain injury. J Neurosurg 73:889-900.
Kihara T, Shimohama S, Sawada H, Honda K, Nakamizo T, Kanki R, Yamashita H, Akaike A. 2002. Protective effect of dopamine D2 agonists in cortical neurons via the phosphatidylinositol 3 kinase cascade. J Neurosci Res 70:274-882.
Kipnis, J., T. Mizrahi, E. Hauben, I. Shaked, E. Shevach, and M. Schwartz. 2002a. Neuroprotective autoimmunity: naturally occurring CD4+CD25+ regulatory T cells suppress the ability to withstand injury to the central nervous system. Proc Natl Acad Sci USA 99:15620-15625.
Kipnis J, Mizrahi T, Yoles E, Ben-Nun A, Schwartz M, Ben-Nur A. 2002b Myelin specific Th1 cells are necessary for post-traumatic protective autoimmunity. J Neuroimmunol. 130(1-2):78-85.
Kirtland, H. H., 3rd, D. N. Mohler, and D. A. Horwitz. 1980. Methyldopa inhibition of suppressor-lymphocyte function: a proposed cause of autoimmune hemolytic anemia. N Engl J Med 302:825-832.
Kleinberg D L, Boyd A E 3rd, Wardlaw S, Frantz A G, George A, Bryan N, Hilal S, Greising J, Hamilton D, Seltzer T, Sommers C J. 1983. Pergolide for the treatment of pituitary tumors secreting prolactin or growth hormone. N Engl J Med 309:704-709.
Kohm, A. P., P. A. Carpentier, H. A. Anger, and S. D. Miller. 2002. Cutting edge: CD4+CD25+ regulatory T cells suppress antigen-specific autoreactive immune responses and central nervous system inflammation during active experimental autoimmune encephalomyelitis. J Immunol 169:4712-4716.
Kukreja A, Cost G, Marker J, Zhang C, Sun Z, Lin-Su K, Ten S, Sanz M, Exley M, Wilson B, Porcelli S, Maclaren N. 2002. Multiple immuno-regulatory defects in type-1 diabetes. J Clin Invest 109(1):131-40.
Lasley, S. M., and M. E. Gilbert. 1996. Presynaptic glutamatergic function in dentate gyms in vivo is diminished by chronic exposure to inorganic lead. Brain Res 736:125-134.
Lee, S. J., and E. N. Benveniste. 1999. Adhesion molecule expression and regulation on cells of the central nervous system. J Neuroimmunol 98:77-88.
Levite, M., Y. Chowers, Y. Ganor, M. Besser, R. Hershkovits, and L. Cahalon. 2001. Dopamine interacts directly with its D3 and D2 receptors on normal human T cells, and activates beta1 integrin function. Eur J Immunol 31:3504-3512.
Liuzzi A, Dallabonzana D, Oppizzi G, Verde G G, Cozzi R, Chiodini P, Luccarelli G. 1985. Low doses of dopamine agonists in the long-term treatment of macroprolactinomas. N Engl J Med 313:656-659.
Liyanage U K, Moore T T, Joo H G, Tanaka Y, Herrmann V, Doherty G, Drebin J A, Strasberg S M, Eberlein T J, Goedegebuure P S, Linehan D C. 2002. Prevalence of regulatory T cells is increased in peripheral blood and tumor microenvironment of patients with pancreas or breast adenocarcinoma. J Immunol 169:2756-2761.
Lohse, A. W., M. Dinkelmann, M. Kimmig, J. Herkel, and K. H. Meyer zum Buschenfelde. 1996. Estimation of the frequency of self-reactive T cells in health and inflammatory diseases by limiting dilution analysis and single cell cloning. J Autoimmun 9:667-675.
Malcangio, M., M. S. Ramer, T. J. Boucher, and S. B. McMahon. 2000. Intrathecally injected neurotrophins and the release of substance P from the rat isolated spinal cord. Eur J Neurosci 12:139-144.
Maloy, K., L. Salaun, R. Cahill, G. Dougan, N. Saunders, and F. Powrie. 2003. CD4+CD25+T(R) cells suppress innate immune pathology through cytokine-dependent mechanisms. J Exp Med 197:111-119.
McHugh, R. S., M. J. Whitters, C. A. Piccirillo, D. A. Young, E. M. Shevach, M. Collins, and M. C. Byrne. 2002. CD4(+)CD25(+) immunoregulatory T cells: gene expression analysis reveals a functional role for the glucocorticoid-induced TNF receptor. Immunity 16:311-323.
Mizrahi, T., E. Hauben, and M. Schwartz. 2002. The tissue-specific self-pathogen is the protective self-antigen: the case of uveitis. J Immunol 169:5971-5977.
Moalem, G., R. Leibowitz-Amit, E. Yoles, F. Mor, I. R. Cohen, and M. Schwartz. 1999. Autoimmune T cells protect neurons from secondary degeneration after central nervous system axotomy. Nat Med 5:49-55.
Mocsai, A., Z. Jakus, T. Vantus, G. Berton, C. A. Lowell, and E. Ligeti. 2000. Kinase pathways in chemoattractant-induced degranulation of neutrophils: the role of p38 mitogen-activated protein kinase activated by Src family kinases. J Immunol 164:4321-4331.
Muller, N., M. Riedel, R. Gruber, M. Ackenheil, and M. J. Schwarz. 2000. The immune system and schizophrenia. An integrative view. Ann N Y Acad Sci 917:456-467.
Nakamura, K., A. Kitani, and W. Strober. 2001. Cell contact-dependent immunosuppression by CD4(+)CD25(+) regulatory T cells is mediated by cell surface-bound transforming growth factor beta. J Exp Med 194:629-644.
Newcomer, J. W., N. B. Farber, V. Jevtovic-Todorovic, G. Selke, A. K. Melson, T. Hershey, S. Craft, and J. W. Olney. 1999. Ketamine-induced NMDA receptor hypofunction as a model of memory impairment and psychosis. Neuropsychopharmacology 20:106-118.

Olsson M, Nikkhah G, Bentlage C, Bjorklund A. 1995. Forelimb akinesia in the rat Parkinson model: differential effects of dopamine agonists and nigral transplants as assessed by a new stepping test. J Neurosci 15:3863-75.
Pasare, C., and R. Medzhitov. 2003. Toll pathway-dependent blockade of CD4+CD25+ T cell-mediated suppression by dendritic cells. Science 299:1033-1036.
Perez, O. D., and G. P. Nolan. 2002. Simultaneous measurement of multiple active kinase states using polychromatic flow cytometry. Nat Biotechnol 20:155-162.
Piccirillo, C. A., J. J. Letterio, A. M. Thornton, R. S. McHugh, M. Mamura, H.
Mizuhara, and E. M. Shevach. 2002. CD4(+)CD25(+) regulatory T cells can mediate suppressor function in the absence of transforming growth factor beta1 production and responsiveness. J Exp Med 196:237-246.
Pozzi, L., K. Hakansson, A. Usiello, A. Borgkvist, M. Lindskog, P. Greengard, and G. Fisone. 2003. Opposite regulation by typical and atypical anti-psychotics of ERK1/2, CREB and Elk-1 phosphorylation in mouse dorsal striatum. J Neurochem 86:451-459.
Ramsdell, F. 2003. Foxp3 and natural regulatory T cells: key to a cell lineage? Immunity 19:165-168.
Ricci, A., S. Mariotta, S. Greco, and A. Bisetti. 1997. Expression of dopamine receptors in immune organs and circulating immune cells. Clin Exp Hypertens 19:59-71.
Rothblat, D. S., and J. S. Schneider. 1998. Effects of GM1 ganglioside treatment on dopamine innervation of the striatum of MPTP-treated mice. Ann N Y Acad Sci 845:274-277.
Rothstein, J. 1995-96. Excitotoxicity and neurodegeneration in amyotrophic lateral sclerosis. Clin Neurosci 3:348-359.
Saha, B., A. C. Mondal, S. Basu, and P. S. Dasgupta. 2001. Circulating dopamine level, in lung carcinoma patients, inhibits proliferation and cytotoxicity of CD4+ and CD8+ T cells by D1 dopamine receptors: an in vitro analysis. Int Immunopharmacol 1:1363-1374.
Sakaguchi, S., N. Sakaguchi, M. Asano, M. Itoh, and M. Toda. 1995. Immunologic self-tolerance maintained by activated T cells expressing IL-2 receptor alpha-chains (CD25). Breakdown of a single mechanism of self-tolerance causes various autoimmune diseases. J Immunol 155:1151-1164.
Sakaguchi, S., T. Takahashi, S. Yamazaki, Y. Kuniyasu, M. Itoh, N. Sakaguchi, and J. Shimizu. 2001. Immunologic self tolerance maintained by T-cell-mediated control of self-reactive T cells: implications for autoimmunity and tumor immunity. Microbes Infect 3:911-918.
Saxena A K, 2002. Dopamine “renal-dose”: an appraisal of current concepts, controversies and concerns. Dialysis & Transplantation 31: 615-621.
Schneider, H., D. A. Mandelbrot, R. J. Greenwald, F. Ng, R. Lechler, A. H. Sharpe, and C. E. Rudd. 2002. Cutting edge: CTLA-4 (CD152) differentially regulates mitogen-activated protein kinases (extracellular signal-regulated kinase and c-Jun N-terminal kinase) in CD4+ T cells from receptor/ligand-deficient mice. J Immunol 169:3475-3479.
Schori, H., J. Kipnis, E. Yoles, E. WoldeMussie, G. Ruiz, L. A. Wheeler, and M. Schwartz. 2001a Vaccination for protection of retinal ganglion cells against death from glutamate cytotoxicity and ocular hypertension: Implications for glaucoma. Proc Natl Acad Sci USA 98:3398-3403.
Schori, H., E. Yoles, and M. Schwartz. 2001b T-cell-based immunity counteracts the potential toxicity of glutamate in the central nervous system. J Neuroimmunol 119:199-204.
Schori, H., E. Yoles, L. A. Wheeler, T. Raveh, A. Kimchi, and M. Schwartz. 2002. Immune-related mechanisms participating in resistance and susceptibility to glutamate toxicity. Eur J Neurosci 16:557-564.
Schwartz, M. and Kipnis, J. 2001. Protective autoimmunity: regulation and prospects for vaccination after brain and spinal cord injuries. Trends Mol Med 7: 252-8.
Schwartz, M. & Kipnis, J. 2002. Autoimmunity on alert: naturally occurring regulatory CD4(+)CD25(+) T cells as part of the evolutionary compromise between a ‘need’ and a ‘risk’. Trends Immunol 23, 530-534.
Sebastiani, S., P. Allavena, C. Albanesi, F. Nasorri, G. Bianchi, C. Traidl, S. Sozzani, G. Girolomoni, and A. Cavani. 2001. Chemokine receptor expression and function in CD4+ T lymphocytes with regulatory activity. J Immunol 166:996-1002.
Sebastiani, S., C. Albanesi, P. O. De, P. Puddu, A. Cavani, and G. Girolomoni. 2002. The role of chemokines in allergic contact dermatitis. Arch Dermatol Res 293:552-559.
Sharp, L. L., D. A. Schwarz, C. M. Bott, C. J. Marshall, and S. M. Hedrick. 1997. The influence of the MAPK pathway on T cell lineage commitment. Immunity 7:609-618,
Shevach, E. M., R. S. McHugh, A. M. Thornton, C. Piccirillo, K. Natarajan, and D. H. Margulies. 2001. Control of autoimmunity by regulatory T cells. Adv Exp Med Biol 490:21-32.
Shimizu, J., S. Yamazaki; and S. Sakaguchi. 1999. Induction of tumor immunity by removing CD25+CD4+ T cells: a common basis between tumor immunity and autoimmunity. J Immunol 163:521.1-5218.
Shimizu, J., S. Yamazaki, T. Takahashi, Y. Ishida, and S. Sakaguchi. 2002. Stimulation of CD25(+)CD4(+) regulatory T cells through GITR breaks immunological self-tolerance. Nat Immunol 3:135-142.
Sundstedt, A., E. O′Neill, K. Nicolson, and D. Wraith. 2003. Role for IL-10 in suppression mediated by peptide-induced regulatory T cells in vivo. J Immunol 170:1240-1248.
Sutmuller R P, van Duivenvoorde L M, van Elsas A, Schumacher T N, Wildenberg M E, Allison J P, Toes R E, Offring a R, Melief C J. 2001. Synergism of cytotoxic T lymphocyte-associated antigen 4 blockade and depletion of CD25(+) regulatory T cells in antitumor therapy reveals alternative pathways for suppression of autoreactive cytotoxic T lymphocyte responses. J Exp Med 194(6):823-32 .
Swanson, M. A., W. T. Lee, and V. M. Sanders. 2001. IFN-gamma production by Th1 cells generated from naïve CD4+ T cells exposed to norepinephrine. J Immunol 166:232-240.
Takahashi, T., T. Tagami, S. Yamazaki, T. Uede, J. Shimizu, N. Sakaguchi, T. W. Mak, and S. Sakaguchi. 2000. Immunologic self-tolerance maintained by CD25(+)CD4(+) regulatory T cells constitutively expressing cytotoxic T lymphocyte-associated antigen 4. J Exp Med 192:303-310.
Takeuchi, Y., and K. Fukunaga. 2003. Differential regulation of NF-kappaB, SRE and CRE by dopamine D1 and D2 receptors in transfected NG108-15 cells. J Neurochem 85:729-739.
Tanimura, S., K. Asato, S. H. Fujishiro, and M. Kohno. 2003. Specific blockade of the ERK pathway inhibits the invasiveness of tumor cells: down-regulation of matrix metalloproteinase-3/-9/-14 and CD44. Biochem Biophys Res Commun 304:801-806.
Teunis, M. A., A. Kavelaars, E. Voest, J. M. Bakker, B. A. Ellenbroek, A. R. Cools, and C. J. Heijnen. 2002. Reduced tumor growth, experimental metastasis formation, and angiogenesis in rats with a hyperreactive dopaminergic system. Faseb J 16:1465-1467.
Thiffault, C., J. W. Langston, and D. A. Di Monte. 2000. Increased striatal dopamine turnover following acute administration of rotenone to mice. Brain Res 885:283-288.
Thornton, A. M., and E. M. Shevach. 1998. CD4+CD25+ immunoregulatory T cells suppress polyclonal T cell activation in vitro by inhibiting interleukin 2 production. J Exp Med 188:287-296.
Thornton, A. M., and E. M. Shevach. 2000. Suppressor effector function of CD4+CD25+ immunoregulatory T cells is antigen nonspecific. J Immunol 164:183-190.
Urushitani, M., S. Shimohama, T. Kihara, H. Sawada, A. Akaike, M. Ibi, R.
Inoue, Y. Kitamura, T. Taniguchi, and J. Kimura. 1998. Mechanism of selective motor neuronal death after exposure of spinal cord to glutamate: involvement of glutamate-induced nitric oxide in motor neuron toxicity and nonmotor neuron protection. Ann Neurol 44:796-807.
Wightman, R. M., and D. L. Robinson. 2002. Transient changes in mesolimbic dopamine and their association with ‘reward’. J Neurochem 82:721-735.
Xiong, H., L. McCabe, D. Skifter, D. T. Monaghan, and H. E. Gendelman. 2003. Activation of NR1a/NR2B receptors by monocyte-derived macrophage secretory products: implications for human immunodeficiency virus type one-associated dementia. Neurosci Lett 341:246-250.
Yi, A. K., J. G. Yoon, S. J. Yeo, S. C. Hong, B. K. English, and A. M. Krieg. 2002. Role of mitogen-activated protein kinases in CpG DNA-mediated IL-10 and IL-12 production: central role of extracellular signal-regulated kinase in the negative feedback loop of the CpG DNA-mediated Th1 response. J Immunol 168:4711-4720.
Yoles, E., and Schwartz, M. 1998. Degeneration of spared axons following partial white matter lesion: implications for optic nerve neuropathies. Exp Neurol 153:1-7.
Yoles, E., E. Hauben, O. Palgi, E. Agranov, A. Gothilf, A. Cohen, V. Kuchroo, I. R. Cohen, H. Weiner, and M. Schwartz. 2001. Protective autoimmunity is a physiological response to CNS trauma. J Neurosci 21:3740-3748.

Zelenika D, Adams E, Humm S, Lin C Y, Waldmann H, Cobbold S P. 2001. The role of CD4+ T-cell subsets in determining transplantation rejection or tolerance. Immunol Rev 182:164-79.

1. A method for modulating the suppressive effect of CD4⁺CD25⁺ regulatory T cells (Treg) on CD4⁺CD25⁻ effector T cells (Teff), which comprises administering to an individual in need an agent selected from the group consisting of dopamine, a dopamine precursor, a dopamine agonist, a dopamine antagonist, and a combination thereof.

2. A method according to claim 1 for down-regulating the suppressive effect of Treg on Teff, said method comprising administering to an individual in need an agent that down-regulates the suppressive activity of Treg on Teff, wherein said agent is selected from the group consisting of:
(i) dopamine or a pharmaceutically acceptable salt thereof;
(ii) a dopamine precursor or a pharmaceutically acceptable salt thereof;
(iii) an agonist of the dopamine receptor type 1 family (D1-R agonist) or a pharmaceutically acceptable salt thereof;
(iv) an antagonist of the dopamine receptor type 2 family (D2-R antagonist) or a pharmaceutically acceptable salt thereof;
(v) a combination of (i) and (ii); and
(vi) a combination of (i), (ii) or (iii) with (iv);
provided that said individual is need is not being treated for a neurodegenerative condition, disorder or disease.

3. A method according to claim 2, wherein said agent is dopamine or a pharmaceutically acceptable salt thereof.

4. A method according to claim 2, wherein said agent is a combination of dopamine and the dopamine precursor levodopa, optionally in further combination with carbidopa.

5. A method according to claim 2, wherein said agent is a dopamine D1-R agonist.

6. The method according to claim 5, wherein said dopamine D1-R agonist is selected from the group consisting of A-77636, SKF-38393, SKF-77434, SKF-81297, SKF-82958, dihydrexidine and fenoldopam.

7. A method according to claim 2, wherein said agent is a dopamine D2-R antagonist.

8. The method according to claim 7, wherein said dopamine D2-R antagonist is selected from the group consisting of amisulpride, clozapine, domperidone, eticlopride, haloperidol, iloperidone, mazapertine, olanzapine, raclopride, remoxipride, risperidone, sertindole, spiperone, spiroperidol, sulpride, tropapride, zetidoline, CP-96345, LU111995, SDZ-HDC-912, and YM 09151-2.

9. A method according to claim 2, wherein said agent is a combination of dopamine with a dopamine D2-R antagonist.

10. The method according to claim 9, wherein said agent is a combination of dopamine with clozapine.

11. A method according to claim 2, wherein said agent is a combination of a dopamine D1-R agonist with a dopamine D2-R antagonist.

12. The method according to claim 11, wherein said agent is a combination of SKF-38393 and clozapine.

13. A method for treatment of cancer,
said method comprising administering to a cancer patient an agent that down-regulates the suppressive activity of CD4⁺CD25⁺ regulatory T cells (Treg) on CD4⁺CD25⁻ effector T cells (Teff), wherein said agent is selected from the group consisting of:
(i) dopamine or a pharmaceutically acceptable salt thereof;
(ii) a dopamine precursor or a pharmaceutically acceptable salt thereof;
(iii) an agonist of the dopamine receptor type 1 family (D1-R agonist) or a pharmaceutically acceptable salt thereof;
(iv) an antagonist of the dopamine receptor type 2 family (D2-R antagonist) or a pharmaceutically acceptable salt thereof;
(v) a combination of (i) and (ii); and
(vi) a combination of (i), (ii) or (iii) with (iv).

14. A method according to claim 13 wherein said agent triggers tumor regression, stimulates the natural immunological defense against cancer, or inhibits cancer cell metastasis.

15. The method according to claim 14 wherein said tumor is a solid tumor.

16. The method according to claim 15 wherein said solid tumor is bladder, brain, breast, cervix, colon, esophagus, head and neck, larynx, liver, lung, melanoma, ovary, pancreas, prostate, renal, stomach, thyroid, uterus, vagina or vocal cord tumor.

17. The method according to claim 15 wherein said tumor is a non-solid malignant neoplasm.

18. The method according to claim 17 wherein said non-solid malignant neoplasma is a lymphoproliferative disorder selected from the group consisting of multiple myeloma, non-Hodgkin's lymphomas, and a lymphocytic leukemia, e.g., chronic lymphocytic leukemia (CLL), prolymphocytic leukemia (PLL), hairy cell leukemia, large granular lymphocyte leukemia and Waldenstrom's macroglubulinemia.

19. A method according to claim 13, wherein said agent is dopamine or a pharmaceutically acceptable salt thereof.

20. A method according to claim 13, wherein said agent is a combination of dopamine and the dopamine precursor levodopa, optionally in further combination with carbidopa.

21. A method according to claim 13, wherein said agent is a dopamine D1-R agonist.

22. The method according to claim 21, wherein said dopamine D1-R agonist is selected from the group consisting of A-77636, SKF-38393, SKF-77434, SKF-81297, SKF-82958, dihydrexidine and fenoldopam.

23. A method according to claim 13, wherein said agent is a dopamine D2-R antagonist.

24. The method according to claim 23, wherein said dopamine D2-R antagonist is selected from the group consisting of amisulpride, clozapine, domperidone, eticlopride, haloperidol, iloperidone, mazapertine, olanzapine, raclopride, remoxipride, risperidone, sertindole, spiperone, spiroperidol, sulpride, tropapride, zetidoline, CP-96345, LU111995, SDZ-HDC-912, and YM 09151-2.

25. A method according to claim 13, wherein said agent is a combination of dopamine with a dopamine D2-R antagonist.

26. The method according to claim 25, wherein said agent is a combination of dopamine with clozapine.

27. A method according to claim 13, wherein said agent is a combination of a dopamine D1-R agonist with a dopamine D2-R antagonist.

28. The method according to claim 27, wherein said agent is a combination of SKF-38393 and clozapine.

29. A method according to claim 1 for up-regulating the suppressive effect of Treg on Teff, said method comprising administering to an individual in need an agent that up-regulates the suppressive activity of Treg on Teff, wherein said agent is selected from the group consisting of:
(i) an antagonist of the dopamine receptor type 1 family (D1-R antagonist) or a pharmaceutically acceptable salt thereof;
(ii) an agonist of the dopamine receptor type 2 family (D2-R agonist) or a pharmaceutically acceptable salt thereof; and
(iii) a combination of (i) and (ii).

30. A method according to claim 29, wherein said agent is a dopamine D2-R agonist or a pharmaceutically acceptable salt thereof.

31. A method according to claim 30, wherein said dopamine D2-R agonist is selected from the group consisting of bromocriptine, cabergoline, lisuride, pergolide, pramipexole, quinagolide, quinpirole, quinelorane, ropinirole, roxindole, talipexole, LY 171555, PPHT and TNPA.

32. A method according to claim 29, wherein said agent is a dopamine D1-R antagonist or a pharmaceutically acceptable salt thereof.

33. A method according to claim 32, wherein said dopamine D1-R antagonist is selected from the group consisting of SCH 23390, NNC 756, NNC 01-112 and CEE-03-310.

34. A method according to claim 29, wherein said agent is a combination of a dopamine D2-R agonist and a dopamine D1-R antagonist.

35. A method for treatment of an autoimmune disease, said method comprising administering to an individual suffering from an autoimmune disease an agent that up-regulates the suppressive activity of CD4⁺CD25⁺+regulatory T cells (Treg) on CD4⁺CD25⁻ effector T cells (Teff), wherein said agent is selected from the group consisting of (i) a dopamine D2-R agonist or a pharmaceutically acceptable salt thereof, a dopamine D1-R antagonist or a pharmaceutically acceptable salt thereof, and (iii) a combination of (i) and (ii).

36. A method according to claim 35, wherein said agent is a dopamine D2-R agonist or a pharmaceutically acceptable salt thereof.

37. A method according to claim 36, wherein said dopamine D2-R agonist is selected from the group consisting of bromocriptine, cabergoline, lisuride, pergolide, pramipexole, quinagolide, quinpirole, quinelorane, ropinirole, roxindole, talipexole, LY 171555, PPHT and TNPA.

38. A method according to claim 35, wherein said agent is a dopamine D1-R antagonist or a pharmaceutically acceptable salt thereof.

39. A method according to claim 38, wherein said dopamine D1-R antagonist is selected from the group consisting of SCH 23390, NNC 756, NNC 01-112 and CEE-03-310.

40. A method according to claim 35, wherein said agent is a combination of a dopamine D2-R agonist and a dopamine D1-R antagonist.

41. A method according to claim 35, wherein said autoimmune disease is Eaton-Lambert syndrome, Goodpasture's syndrome, Grave's disease, Guillain-Barré syndrome, autoimmune hemolytic anemia (AIHA), hepatitis, insulin-dependent diabetes mellitus (IDDM), systemic lupus erythematosus (SLE), multiple sclerosis (MS), myasthenia gravis, plexus disorders, e.g., acute brachial neuritis, polyglandular deficiency syndrome, primary biliary cirrhosis, rheumatoid arthritis, scleroderma, thrombocytopenia, thyroiditis, e.g., Hashimoto's disease, Sjögren's syndrome, allergic purpura, psoriasis, mixed connective tissue disease, polymyositis, dermatomyositis, vasculitis, polyarteritis nodosa, polymyalgia rheumatica, Wegener's granulomatosis, Reiter's syndrome, Behçet's syndrome, ankylosing spondylitis, pemphigus, bullous pemphigoid, dermatitis herpetiformis, Crohn's disease or uveitis.

42. A method for controlling graft rejection in an individual undergoing tissue or organ transplantation which comprises administering to said individual an agent that up-regulates the suppressive activity of CD4⁺CD25⁺ regulatory T cells (Treg) on CD4⁺CD25⁻ effector T cells (Teff), wherein said agent is selected from the group consisting of (i) a dopamine D2-R agonist or a pharmaceutically acceptable salt thereof, a dopamine D1-R antagonist or a pharmaceutically acceptable salt thereof, and a combination of (i) and (ii).

43. A method according to claim 42, wherein said agent is a dopamine D2-R agonist or a pharmaceutically acceptable salt thereof.

44. A method according to claim 43, wherein said dopamine D2-R agonist is selected from the group consisting of bromocriptine, cabergoline, lisuride, pergolide, pramipexole, quinagolide, quinpirole, quinelorane, ropinirole, roxindole, talipexole, LY 171555, PPHT and TNPA.

45. A method according to claim 42, wherein said agent is a dopamine D1-R antagonist or a pharmaceutically acceptable salt thereof.

46. A method according to claim 45, wherein said dopamine D1-R antagonist is selected from the group consisting of SCH 23390, NNC 756, NNC 01-112 and CEE-03-310.

47. A method according to claim 42, wherein said agent is a combination of a dopamine D2-R agonist and a dopamine D1-R antagonist.

48-122. (canceled)


SEQUENCE LISTING (20 sequences)

SEQ ID NO: 1 - DNA, 20 bp, Artificial Sequence
  Synthetic
gtagccattatgatcgtcac

SEQ ID NO: 2 - DNA, 21 bp, Artificial Sequence
  Synthetic
gatcacagacagtgtcttcag

SEQ ID NO: 3 - DNA, 20 bp, Artificial Sequence
  Synthetic
gcagccgagctttcagggcc

SEQ ID NO: 4 - DNA, 20 bp, Artificial Sequence
  Synthetic
gggatgttgcagtcacagtg

SEQ ID NO: 5 - DNA, 18 bp, Artificial Sequence
  Synthetic
aggtttctgtcagatgcc

SEQ ID NO: 6 - DNA, 19 bp, Artificial Sequence
  Synthetic
gttgctgagttttcgaacc

SEQ ID NO: 7 - DNA, 20 bp, Artificial Sequence
  Synthetic
caccaactacttcatcgtga

SEQ ID NO: 8 - DNA, 20 bp, Artificial Sequence
  Synthetic
aaggagcagacggacgagta

SEQ ID NO: 9 - DNA, 19 bp, Artificial Sequence
  Synthetic
ctacgagcgcaagatgacc

SEQ ID NO: 10 - DNA, 19 bp, Artificial Sequence
  Synthetic
ctctgagcatgctcagctg

SEQ ID NO: 11 - DNA, 32 bp, Artificial Sequence
  Synthetic
gtgcagtcctgaaggacttcaagctccaccag

SEQ ID NO: 12 - DNA, 30 bp, Artificial Sequence
  Synthetic
ggcaaggaccctgacctatggggtcatcac

SEQ ID NO: 13 - DNA, 22 bp, Artificial Sequence
  Synthetic
cagctgcctacagtgcccctag

SEQ ID NO: 14 - DNA, 20 bp, Artificial Sequence
  Synthetic
catttgccagcagtgggtag

SEQ ID NO: 15 - DNA, 16 bp, Artificial Sequence
  Synthetic
cctttatcccggtcca

SEQ ID NO: 16 - DNA, 17 bp, Artificial Sequence
  Synthetic
gatacggcggatctgaa

SEQ ID NO: 17 - DNA, 26 bp, Artificial Sequence
  Synthetic
acctggtagaagtgatgccccaggca

SEQ ID NO: 18 - DNA, 25 bp, Artificial Sequence
  Synthetic
ctatgcagttgatgaagatgtcaaa

SEQ ID NO: 19 - DNA, 22 bp, Artificial Sequence
  Synthetic
cagctgcctacagtgcccctag

SEQ ID NO: 20 - DNA, 20 bp, Artificial Sequence
  Synthetic
catttgccagcagtgggtag